(19) United States
(12) Patent Application Publication
(10) Pub. No.: US 2014/0272974 A1
(43) Pub. Date: Sep. 18, 2014

(54) NOVEL RETROELEMENT FOUND IN MOLLUSKS

(71) Applicant: The Trustees of Columbia University in the City of New York, New York, NY (US)

(72) Inventors: Stephen P. Goff, New York, NY (US); W. Ian Lipkin, New York, NY (US); Gloria Arriagada, Las Condes (CL); Carol Reinisch, Falmouth, MA (US); James Sherry, Hamilton (CA); Charles Walker, Barrington, NH (US)

(73) Assignee: The Trustees of Columbia University in the City of New York, New York, NY (US)

(21) Appl. No.: 14/215,488

(22) Filed: Mar. 17, 2014

Related U.S. Application Data
(60) Provisional application No. 61/799,791, filed on Mar. 15, 2013.

(51) Int. Cl. C12Q 1/68; G01N 33/574

(52) U.S. Cl. CPC C12Q 1/6886; G01N 33/57496
USPC 435/6.11; 536/23.5; 435/320.1; 530/387.9; 435/7.1

(57) ABSTRACT

This invention relates to a novel retroelement, named “Steamer”, found in mollusks, more specifically Mya arenaria, that is associated with haemic neoplasia in these organisms. Haemic neoplasia (HN) is a recognizable leukemic-like disease.

The invention provides the retroelement protein, antibodies to the protein, nucleic acids encoding the protein, probes, primer, gene constructs comprising the nucleic acids, host cells comprising the nucleic acids, and methods of using.

### CROSS REFERENCE TO RELATED APPLICATION

The present application claims priority to U.S. patent application Ser. No. 61/799,791 filed Mar. 15, 2013, which is hereby incorporated by reference in its entirety.

### FIELD OF THE INVENTION

This invention relates to a novel retroelement, named “Steamer”, found in mollusks, more specifically Mya arenaria, that is associated with haemic neoplasia in these organisms. Haemic neoplasia (HN) is a recognizable leukemic-like disease.

The invention provides the retroelement protein, antibodies to the protein, nucleic acids encoding the protein, probes, primers, gene constructs comprising the nucleic acids, host cells comprising the nucleic acids, and methods of using.

### BACKGROUND OF THE INVENTION

The Atlantic soft-shell clam, Mya arenaria, is a bivalve mollusk is native to the Atlantic Coast of North America and inhabits a range extending from Maryland to Canada. The commercial harvest is economically significant (about $15 million per annum). Over the past thirty years the species has been subject to a neoplastic disease of rapidly increasing prevalence, known as “hematopoietic neoplasia”, “disseminated neoplasia” (DN) or “haemic neoplasia” (HN) (Barber (2004); Cooper et al. (1982); Elston et al. (1992); Farley et al. (1986); Morrison et al. (1993)). The beds in many locations have been decimated by the disease, and the incidence in affected areas can range from 10% to as high as 90% of the animals (Brown et al. (1977)). The disease is similar in many ways to mammalian leukemia, with a huge expansion of blast-like cells in the hemolymph with high mitotic index (Smolowitz et al. (1989)). The cells are polyploid/aneuploid (Cooper et al. (1982); Lowe and Moore (1978); Reno et al. (1994)), and often express a novel 200-kD cell surface antigen as defined by a 1e10 monoclonal antibody (Miosky et al. (1989); Reinisch et al. (1983); Smolowitz and Reinisch (1993); White et al. (1993)). The p53 tumor suppressor protein (Holbrook et al. (2009); Kelley et al. (2001); St.-Jean et al. (2005); Walker et al. (2006)) is expressed in the tumor cells, but is sequestered out of the nucleus and into the cytoplasm by binding the mitochondrial heat shock protein mortalin (Barker et al. (1997); Bottger et al. (2008); Walker et al. 2006)). A similar disease has been described in several species of bivalves, including oysters (Crassostrea virginica, C. gigas, Ostrea eduli), mussels (Mytilus edulis, M. galloprovincialis, M. trossulus, M. chilensis), cockles (Cerastoderma edule), and clams (Macoma spp., Mya arenaria, and M. trunata) over a wide geographic distribution.

Despite many reported clinical cases, the etiology of the disease is mysterious (Barber (2004); Muttray et al. (2012)). Suggestions have included both environmental pollution (Landsberg (1996)), temperature (Schneider (2008)), and infectious agents (Collins and Mulcahy (2003); Oprandy et al. (1981)). Experimental transmission of disease between animals by cells or cell-free hemolymph has been reported (Sunila (1992)) but not consistently verified. Reverse transcriptase activity in tissues and hemolymph has been sporadically reported (AboElkhair et al. (2009); AboElkhair et al. (2009); House et al. (1998)), and very recently, increased levels of retrovirus-related RNAs have been detected by Q-PCR with generic viral primers (Siah et al. (2011)). However, to date no viruses or retroviral sequences from leukemic clams has been identified (AboElkhair et al. (2012)).

This disease of the mollusk Mya arenaria, is inherently interesting. The host organism has been suggested to serve as a “canary in the coal mine” as a reporter of environmental stresses and pollution. This is a rare model of a “leukemia in the wild” that is in epidemic growth, and has no clear etiology. The leukemia may be associated with environmental contamination, with disease clearly arising in clusters at specific geographic locations (Krishnakumar et el. (1999)), but it may also be associated with an infectious agent.

Leukemic clams are routinely found at specific sites in Prince Edward Island, while other sites are completely disease-free. The organism has many attractive features: the animals are relatively easy to collect, they can be maintained in the laboratory, and cells can be cultured in relatively conventional tissue culture medium (Sunila and Farley (1989)). This is perhaps one of the most primitive organisms with a recognizable leukemia-like disease. The sequencing of the genome has just been completed, and candidate genes of likely involvement are easily identified by their similarity to the mammalian orthologues. Oncogenes and tumor suppressor genes such as p53 are present (Kelley et al. (2001); St.-Jean et al. (2005); Walker et al. (2011)), and indeed abnormalities in p53 levels and localization have been noted in the tumor cells.

To date there is no large-scale inexpensive test for HN in clam harvests. Current technology is to test clam samples for disease by histological test by microscopic observation of hemocytes drawn from animals. This test is limited to small-scale and cannot be readily performed large-scale or simultaneously with other tests. Thus, there is a need for a rapid, inexpensive large-scale test for surveys of large numbers of samples, that can performed simultaneously with similar tests for pathogens.

Additionally, an understanding of the basis of this disease could well inform our understanding of other diseases, such as human leukemia, making this organism an important tool for determination of the causes and development of treatment of human leukemia.

### SUMMARY OF THE INVENTION

The current invention provides a novel retroelement denoted as “steamer,” from mollusks, including functional homologues, derivatives, and fragments. The mollusks can include, but are not limited to, clams, oysters, scallops, mussels, snails, and soft-shelled clams. In a preferred embodiment, the mollusk is the species of soft-shelled clam Mya arenaria.

In a preferred embodiment, the retroelement comprises the polypeptide sequence of SEQ ID NO: 3 as well as functional homologues, derivatives, and fragments of the polypeptide comprising SEQ ID NO: 3.

The current invention also comprises a nucleic acid encoding a novel retroelement denoted as “steamer,” from mollusks, including functional homologues, derivatives, and fragments. The mollusks can include, but are not limited to, clams, oysters, scallops, mussels, snails, and soft-shelled clams. In a preferred embodiment, the mollusk is the species of soft-shelled clam Mya arenaria.

In another embodiment, the DNA of the retroelement comprises the cDNA sequence of SEQ ID NO: 1 as well as functional homologues, derivatives, and fragments of the nucleotide comprising the sequence of SEQ ID NO: 1, and DNA that is complementary, and/or hybridizes to the sequence of SEQ ID NO.: 1 as well as DNA that is complementary, and/or hybridizes functional homologues, derivatives, and fragments of the nucleotide comprising the sequence of SEQ ID NO: 1.

In a further embodiment, the RNA of the retroelement comprises the sequence of SEQ ID NO: 2 as well as functional homologues, derivatives, and fragments of the nucleic acid comprising SEQ ID NO: 2 and RNA that is complementary, and/or hybridizes to the sequence of SEQ ID NO.: 2 as well as RNA that is complementary, and/or hybridizes to functional homologues, derivatives, and fragments of the nucleotide comprising the sequence of SEQ ID NO: 2.

The present invention also provides an antibody directed to a purified mollusk retroelement polypeptide and homologue, derivatives, and fragments thereof.

The present invention also provides for probes and primers comprising the nucleic acid encoding the “steamer” retroelement and homologues, derivatives, and fragments thereof.

The present invention also includes constructs and host cells comprising the steamer retroelement nucleic acid and homologues, derivatives, and fragments thereof.

The present invention also provides for methods of using the steamer retroelement polypeptide, antibodies, nucleic acids, probes, primers, gene constructs, and host cells.

In particular, the present invention provides the use of a nucleic acid of the invention or an antibody of the invention to detect the presence of a mollusk retroelement, which in turn detects or identifies haemic neoplasia in a mollusk. The novel retroelement nucleic acid and antibodies directed to the retroelement can be used to screen and identify neoplasia and leukemia in other subjects.

One embodiment of the present invention is a method or assay for screening and/or identifying neoplasia or leukemia, comprising obtaining biological tissue from a subject, purifying and/or isolating nucleic acid, including, but not limited to, genomic DNA and RNA from the biological tissue, and detecting the presence of the steamer retroelement in the nucleic acid, wherein the presence of the steamer element identifies the subject as having a neoplasia or leukemia.

This embodiment can be a method of, or an assay for identifying or screening for a neoplasia or leukemia in a subject comprising:
a. obtaining a sample of deoxyribonucleic acid or ribonucleic acid from the subject;
b. contacting the sample of step (a) with a nucleic acid that specifically hybridizes with the cDNA of SEQ ID NO: 1, under conditions permitting the nucleic acid to specifically hybridize to a deoxyribonucleic acid or ribonucleic acid encoding a retroelement;
c. detecting any hybridization in step (b), and
d. determining that the subject has a neoplasia or leukemia based upon the binding of the cDNA with the deoxyribonucleic acid or ribonucleic acid encoding a portion of a retroelement in the sample.

In a preferred embodiment, the subject is a mollusk, and a more preferred embodiment the mollusk is a clam, oyster, scallop, mussel, snail, or soft-shelled clams, and in a most preferred embodiment the mollusk is Mya arenaria. It is preferred that the neoplasia being identified is haemic neoplasia.

It is also preferred that the method further comprise providing a healthy control sample, and contacting the cDNA of SEQ ID NO: 1 to obtain a threshold level, wherein the step of determining that the patient has a neoplasia or leukemia comprises a step of comparing the binding to the threshold level, and wherein the binding is greater than the threshold level, the subject is determined to have a neoplasia or leukemia. Again in this embodiment, it is preferred that the subject is a mollusk, and a more preferred embodiment the mollusk is a clam, oyster, scallop, mussel, snail, or soft-shelled clams, and in a most preferred embodiment the mollusk is Mya arenaria. It is also preferred that the healthy control is a mollusk without HN.

This embodiment also comprises the use of primers to amplify DNA and polymerase chain reaction.

The invention also provides for a method of identifying or screening for a neoplasia or leukemia in a subject, comprising:
a. obtaining a sample of cells or protein from the subject;
b. contacting the sample with the antibody of directed to a retroelement found in mollusks and associated with haemic neoplasia;
c. detecting any specific binding in step (b); and
d. determining the subject has a neoplasia or leukemia based upon the binding of the antibody with the retroelement in the sample.

In a preferred embodiment, the subject is a mollusk, and a more preferred embodiment the mollusk is a clam, oyster, scallop, mussel, snail, or soft-shelled clams, and in a most preferred embodiment the mollusk is Mya arenaria. It is preferred that the neoplasia being identified is haemic neoplasia.

It is also preferred that the retroelement to which the antibody is directed comprises the polypeptide comprising the amino acid sequence of SEQ ID NO: 3 or functional homologues, derivatives or fragments thereof.

It is also preferred that the method further comprise providing a healthy control sample, and contacting the antibody directed to a retroelement found in mollusks and associated with haemic neoplasia to obtain a threshold level, wherein the step of determining that the subject has a neoplasia or leukemia comprises a step of comparing the binding to the threshold level, and wherein the binding is greater than the threshold level, the subject is determined to have a neoplasia or leukemia. Again in this embodiment, it is preferred that the subject is a mollusk, and a more preferred embodiment the mollusk is a clam, oyster, scallop, mussel, snail, or soft-shelled clams, and in a most preferred embodiment the mollusk is Mya arenaria. It is also preferred that the healthy control is a mollusk without HN.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1A depicts the autoradiography images of hemolymph from diseased clams (“Leukemic” or “L”) and healthy normal clams (“Normal” or “N”) incubated in reverse transcriptase reactions containing ³²P-TTP and homopolymer substrate (oligo(dT):poly(rA)).

FIG. 1B shows the same experiment as FIG. 1A except using cell culture supernatant.

FIG. 1C shows alignment of selected sequences obtained by deep sequencing of cDNAs from a leukemic clam with a retroviral pol gene. PCR primers, forward (F) and reverse (R), are indicated. DNAs amplified by various primer pairs are indicated below the element diagram.

FIG. 1D depicts the results of PCR and the DNAs amplified in PCR reactions using cDNA obtained from leukemic clams as a template. Major amplified products are indicated by arrows at the right.

FIG. 1E shows a schematic of the Steamer genome annotated with characteristic retroelement features. The 5′ and 3′ LTR and the locations of the coding sequences for CA (capsid), NC (nucleocapsid), PR (protease), RT (reverse transcriptase), RH (RNaseH), and IN (integrase) domains are indicated. Characteristic sequence features of each domain, and predicted primer binding site (PBS) and polypurine track (PPT) are indicated.

FIG. 2 is a Steamer phylogenic tree, a maximum likelihood tree generated by PhyML using the amino acid sequences of the conserved regions of the Gag, Protease, RT, RNase H, and IN domains of Steamer and representative sequences from a database of retrotransposon sequences. Bootstrap values above 75 are shown.

FIG. 3 is a graph depicting the results of quantitative RT-PCR and the relative standard curve method showing levels of Steamer RNA. The results are expressed as relative levels compared to EF1 mRNA and are shown on Y-axis log scale. Each circle, square and triangle represents RNA from a single individual animal. The geometric mean values, indicated by the horizontal line, were compared by two-tailed T test.

FIGS. 4A-C depict Southern blots of total DNA from hemolymph of healthy (N) or diseased (HL) specimens. FIG. 4A shows a schematic representation of the Steamer retrotransposon. LTRs at the 5′ and 3′ ends, Gag-Pol ORF, sites for digestion by the indicated restriction enzymes and location of the ³²P-labeled probe are indicated. Nucleotide positions are relative to the first nucleotide of the U3 portion of the 5′ LTR. FIG. 4B shows a Southern blot of genomic DNA of four normal (Nor1-4) and one heavily leukemic animal (Dnear-HL03) digested with restriction enzymes BamHI, releasing left junction fragments, or with DraI, releasing an internal fragment. FIG. 4C shows a Southern blot of genomic DNA from two normal individuals (Nor1-2) and three leukemic individuals (Dnear-HL03, Dnear-07 and Dnear-08) digested with KpnI, releasing an internal fragment. The migration of the DNA molecular markers is indicated at the left of the panels, and major fragment recognized by the probe is indicated by *.

FIGS. 5A and B show the results of Southern analysis of Steamer DNA analyzed with several digests and two hybridization probes. FIG. 5A is a schematic of the retrotransposon. Positions of selected restriction enzyme digestion sites and two hybridization probes are indicated. FIG. 5B is a Southern blot of DNA from hemocytes of a normal (N) and highly leukemic (HL) clam were digested with enzymes: Lanes 1: BamHI. Lanes 2: DraI. Lanes 3: EcoRI. Lanes 4: HindIII. Blots were hybridized with probe 1 (left panel) or probe 2 (right panel) as indicated. Positions of major internal fragments released from the HL DNA by BamHI, HindIII, and DraI are indicated with arrows. The “noncutter” EcoRI only releases a large smear of DNAs of heterogeneous sizes.

FIGS. 6A-C depict the results of inverse PCR. FIG. 6A is a schematic of inverse PCR methodology: genomic DNA was digested with MfeI (cleaving only in the flanking DNA), circularized by ligation, and redigested with NsiI at internal sites (N), and finally PCR was performed with outward-directed LTR primers. FIG. 6B shows a film of agarose gel electrophoresis of the PCR products of one normal animal (WfarNM01), and two heavily leukemic animals (Dnear-08, Dnear-HL03). For WfarNM01, the white arrowhead marks amplification of the internal Steamer sequence (due to incomplete NsiI cleavage) and the black arrowhead marks the junction product of a single Steamer copy. The leukemic samples (L) yielded a large number of heterogeneous junction products. FIG. 6C depicts representative DNA sequences of individual cloned integration sites from normal and leukemic DNAs. The genomic DNA flanking sequences, the 5 bp duplicated repeats, and the Steamer termini are shown. The presence of the integration sites in the source DNAs was confirmed for each of the sequences shown by a diagnostic PCR using a forward primer in the Steamer LTR and a reverse primer in the flanking genomic DNA (right panels; products are approximately 150 bp).

### DETAILED DESCRIPTION OF THE INVENTION

The current invention comprises a novel retroelement denoted as “steamer,” from mollusks, including functional homologues, derivatives, and fragments. The mollusks can include, but are not limited to, clams, oysters, scallops, mussels, snails, and soft-shelled clams. In a preferred embodiment, the mollusk is the species of soft-shelled clam Mya arenaria.

In a preferred embodiment, the retroelement comprises the polypeptide sequence of SEQ ID NO: 3 as well as functional homologues, derivatives, and fragments of the polypeptide comprising SEQ ID NO: 3.

The current invention also comprises a nucleic acid encoding a novel retroelement denoted as “steamer,” from mollusks, including functional homologues, derivatives, and fragments. The mollusks can include, but are not limited to, clams, oysters, scallops, mussels, snails, and soft-shelled clams. In a preferred embodiment, the mollusk is the species of soft-shelled clam Mya arenaria.

In another embodiment, the DNA of the retroelement comprises the sequence of SEQ ID NO: 1 as well as functional homologues, derivatives, and fragments of the nucleotide comprising the sequence of SEQ ID NO: 1, and DNA that is complementary, and/or hybridizes to the sequence of SEQ ID NO.: 1 as well as functional homologues, derivatives, and fragments of the nucleotide comprising the sequence of SEQ ID NO: 1.

In a further embodiment, the RNA of the retroelement comprises the sequence of SEQ ID NO: 2 as well as functional homologues, derivatives, and fragments of the nucleic acid comprising SEQ ID NO: 2 and RNA that is complementary, and/or hybridizes to the sequence of SEQ ID NO.: 2 as well as functional homologues, derivatives, and fragments of the nucleotide comprising the sequence of SEQ ID NO: 2.

The present invention also provides an antibody directed to a purified mollusk retroelement polypeptide and homologue, derivatives, and fragments thereof.

The present invention also provides for probes and primers comprising the nucleic acid encoding the “steamer” retroelement and homologues, derivatives, and fragments thereof.

The present invention also includes constructs and host cells comprising the steamer retroelement nucleic acid and homologues, derivatives, and fragments thereof.

The present invention also provides for methods of using the steamer retroelement polypeptide, antibodies, nucleic acids, probes, primers, constructs, and host cells.

### DEFINITIONS

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “steamer” or “Steamer” or “steamer retroelement” will be used interchangeably and is the novel retroelement discovered mollusks, which is associated with at least the disease, haemic neoplasia (HN).

The term “subject” as used in this application means an animal. The animal can be an invertebrate such as a mollusk, or a mammal or avian. Mammals include canines, felines, rodents, bovine, equines, porcines, ovines, and primates. Avians include fowls, songbirds, and raptors.

The terms “screen” and “screening” and the like as used herein means to test a subject for the presence of the steamer retroelement or to determine if they have a particular illness or disease. The term also means to test an agent to determine if it has a particular action or efficacy.

The terms “identification”, “identify”, “identifying” and the like as used herein means to recognize the steamer retroelement and/or a disease in a subject. The term also means to recognize an agent as being effective for a particular use.

The term “reference value” as used herein means an amount of a quantity of a particular protein or nucleic acid in a sample from a healthy control.

The term “threshold level” would be the level of binding to a nucleic acid or antibody as seen visually in a healthy control.

The term “healthy control” would be a mollusk without haemic neoplasm or in another animal, one without disease.

The term “agent” as used herein means a substance that produces or is capable of producing an effect and would include, but is not limited to, chemicals, pharmaceuticals, biologics, small organic molecules, antibodies, nucleic acids, peptides, and proteins.

The terms “nucleic acid”, “polynucleotide” and “nucleic acid sequence” are used interchangeably herein, and each refers to a polymer of deoxyribonucleotides and/or ribonucleotides. The deoxyribonucleotides and ribonucleotides can be naturally occurring or synthetic analogues thereof. “Nucleic acid” shall mean any nucleic acid, including, without limitation, DNA, RNA and hybrids thereof. “Nucleotides” shall mean the nucleic acid bases that form nucleic acid molecules and can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, New Jersey, USA). Nucleic acids include, without limitation, antisense molecules and catalytic nucleic acid molecules such as ribozymes and DNAzymes. Nucleic acids also include nucleic acids coding for peptide analogs, fragments or derivatives which differ from the naturally-occurring forms in terms of the identity of one or more amino acid residues (deletion analogs containing less than all of the specified residues; substitution analogs wherein one or more residues are replaced by one or more residues; and addition analogs, wherein one or more resides are added to a terminal or medial portion of the peptide) which share some or all of the properties of the naturally-occurring forms.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, and carbamates) and with charged linkages (e.g., phosphorothioates, and phosphorodithioates). Polynucleotides may contain one or more additional covalently linked moieties, such as, for example, proteins (e.g., nucleases, toxins, antibodies, signal peptides, and poly-L-lysine), intercalators (e.g., acridine, and psoralen), chelators (e.g., metals, radioactive metals, iron, and oxidative metals), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The terms “polypeptide,” “peptide” and “protein” are used interchangeably herein, and each means a polymer of amino acid residues. The amino acid residues can be naturally occurring or chemical analogues thereof. Polypeptides, peptides and proteins can also include modifications such as glycosylation, lipid attachment, sulfation, hydroxylation, and ADP-ribo sylation.

Units, prefixes and symbols may be denoted in their SI accepted form. Unless otherwise indicated, nucleic acid sequences are written left to right in 5′ to 3′ orientation and amino acid sequences are written left to right in amino- to carboxy-terminal orientation. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “homologue” and the like refer to a protein having a having a very similar primary, secondary, and tertiary structure. The term also refers to a nucleic acid with a very similar nucleotide structure.

The term “derivative” and the like is a protein or nucleic acid with a modification.

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid).

As used herein, the term “specifically hybridizes” refers to the ability of a nucleic acid to hybridize to at least 15 consecutive nucleotides of the target sequence, such as a retroelement DNA or RNA, or a sequence complementary thereto, or naturally occurring mutants thereof, such that it has less than 15%, preferably less than 10%, and more preferably less than 5% background hybridization to a non-target nucleic acid.

As used herein, the term “standard hybridization conditions” refers to hybridization conditions that allow hybridization of sequences having at least 75% sequence identity. According to a specific embodiment, hybridization conditions of higher stringency may be used to allow hybridization of only sequences having at least 80% sequence identity, at least 90% sequence identity, at least 95% sequence identity, or at least 99% sequence identity.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “purified” and the like as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include, but are not limited to, plasmids, phages, and viruses.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct” or “gene construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described herein.

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin. Sequence identity can be determined using any of a number of publicly available sequence comparison algorithms, such as BLAST, FASTA, DNA Strider, or GCG (Genetics Computer Group, Program Manual for the GCG Package, Version 7, Madison, Wis.).

The terms “substantially homologous” or “substantially similar” when at least about 80%, and most preferably at least about 90 or 95%, 96%, 97%, 98%, or 99% of the nucleotides match over the defined length of the DNA sequences, as determined by sequence comparison algorithms, such as BLAST, FASTA, and DNA Strider. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### The “Steamer” Retroelement

Haemic neoplasia (HN) is a proliferative cell disorder of the circulatory system of the soft shell clam, Mya arenaria. There is very little information how this leukemia-like disease might be caused. One model for the induction of disease is environmental toxins and a viral “trigger”. There have often been indications of correlation of HN with exposure to toxins, and though the correlations are not perfect, it is plausible that such stresses may promote tumorigenesis. Retroviruses have been proposed as possible etiological agents (Medina et al. (1993)), but efforts to document their detection have been mixed, and recently the possibility of such viruses has been firmly dismissed (AboElkhair et al. (2012)). However, the results herein document the presence of high RT levels and high viral RNA expression in diseased mollusks.

The results herein also show a novel retroelement named “steamer” was found in the hemolymph of diseased mollusks. By extracting RNA from the cell-free hemolymph of mollusks with neoplasms, the cDNA of the retroelement was synthesized (SEQ ID NO: 1). It has also been shown that the retroelement has a single long intact reading frame encoding the predicted Gag-Pol protein with NC, PR, RT, and IN domains of a leukemia virus (SEQ ID NO: 3). Additionally, the results show that the steamer retroelement DNA is highly amplified in diseased clams. Thus, at the very least there is an association between the steamer retroelement and haemic neoplasia.

Transposons, ubiquitous in the genomes of all eukaryotes, are by convention grouped into families based on their sequence similarity. The Steamer element of Mya arenaria is a member of the gypsy/Ty3 family of retrotransposons, which are marked by the presence of LTRs and undergo reverse transcription and integration by mechanisms virtually identical to those used by the true retroviruses (Levin (2002)). The single gene product encoded by Steamer contains many of the motifs present on retrovirus Gag and Pol proteins, including those of the capsid, nucleocapsid, protease, reverse transcriptase, RNase H, and integrase. Steamer does not encode an envelope protein. Most gypsy family members do not encode envelope proteins, and most retrotransposition events mediated by these elements are likely to occur intracellularly, by the formation of cytoplasmic virion-like particles that mediate reverse transcription and DNA integration into the genome of the same cell. Those elements that do encode envelope proteins (such as ZAM (Brasset et al. (2006)) and gypsy itself (Song et al. (1994)) can act as infectious retroviruses and can transmit from cell-to-cell and from one animal to another, perhaps with the help of cellular vesicle trafficking machinery (Brasset et al. (2006); Song et al. (1994); Kim et al. (1994)). But such infection events may take place even without the use of the envelope protein encoded by the element (McLaughlin et al. (1992)) and in these cases an envelope-like protein from the cell, or from a complementing retroelement, may provide the functionality in trans. The filter-feeding mollusks are capable of concentrating viruses present at very low concentrations in seawater, and can concentrate even viruses, such as human hepatitis A virus, that do not replicate in the mollusk, to sufficient levels to allow infection of humans upon ingestion. Thus, though Steamer does not contain an envelope gene, it is easily conceivable that virion-like particles could mediate movement of the element horizontally from one animal to another. This process may explain the accounts of transmission of disease by filtered hemolymph or by co-culture of healthy animals with leukemic animals (Collins and Mulcahy (2003); Oprandy et al. (1981); Walker et al. (2009)).

There is also evidence that the novel “Steamer” retroelement is a new exogenous retrovirus. The virus itself is of considerable interest to retrovirologists, especially those involved in the phylogeny and evolution of the virus family. No one has studied these primitive marine retroviruses before. Perhaps the closest well-studied retroviruses are the piscine (fish) epsilonretroviruses: the walleye dermal sarcoma viruses (Rovnak and Quackenbush (2010)) (notable as encoding their own cyclins), the snakehead fish retrovirus (Hart et al. (1996)), and perhaps a salmon leukemia virus (Eaton and Kent (1992)).

It is possible that activation of Steamer element associated with leukemia may be a consequence rather than a cause of tumor development. A recent study has documented significant changes in the expressed mRNAs of hemocytes from HN animals as compared to healthy animals, suggesting alterations in the transcriptional program that could include Steamer activation (Siah et al. (2013)).

Transposons create insertional mutations upon each transposition event, and thus can be agents of profound genome instability in cancers (Inaki and Liu (2012); Solyom et al. (2012)). The scale of activation of Steamer in leukemic cells seen here is extraordinary, unprecedented in magnitude for an induction of transposition in a natural setting. The introduction of more than 100 new copies of a retroelement per genome is bound to lead to profound genetic changes, and it is very plausible that Steamer activity and amplification is involved as a factor or cofactor in the initial development of the leukemia. There are so many new copies of Steamer DNA per genome in the leukemia cells that it will be hard to determine if there has been an insertional activation of a critical oncogene, but the leukemias are clearly polyclonal with respect to Steamer insertions and are acquiring new proviruses as the pool of transformed cells expands. One or more of the new insertions could significantly alter the phenotypes of these cells.

Endogenous retroviruses and retroelements in mammals are often induced by DNA damaging agents, notably halogenated nucleosides such as bromodeoxyuridine (BrdU) and iododeoxyuridine (IdU), and this induction can be enhanced by polycyclic hydrocarbons (Yoshikura et al. (1977)). Thus, exposures to environmental toxins may be triggers for the activation of Steamer and disease. An induction of Steamer either early or late in the course of disease would induce rapid genetic instability and so could accelerate or promote disease progression. This scenario may account for the ability of BrdU to experimentally induce disease in clams (Oprandy and Chang (1983)

Recent studies have shown that some clam populations are more susceptible than others to induction of disease by DNA damaging agents (Taraska and Bottger (2013)). If Steamer is responsible for the disease, susceptible populations may harbor a higher copy number of Steamer or distinctive copies that are more readily induced for expression. Both inheritance of a high number of endogenous copies of the element and somatic amplification of the element within individuals could contribute to development of disease.

The current invention for the first time allows the availability of steamer cDNA, RNA, and polypeptide sequences for use as probes, primers, and antibodies to allow for large-scale, inexpensive surveys of the prevalence of the element in various populations of mollusks. Additionally, the present invention allows the tests of experimental transmission from animal to animal, and further tests for its functional involvement with disease.

Because genomes of Mya arenaria are highly polymorphic for the Steamer element, the cDNA also allows the development of populations of Mya arenaria that lack the element entirely through selective breeding, and such element-free populations may be less prone to induction of leukemia by environmental stresses.

The identification of Steamer and its dramatic amplification in leukemia provides a new marker for the disease.

### The Steamer Retroelement Nucleic Acid

The present invention provides an isolated polynucleotide comprising all, or a portion of the steamer retroelement present in a mollusk. The mollusk can include, but is not limited to, clams, oysters, scallops, mussels, snails, and soft-shelled clams. In a preferred embodiment, the mollusk is the species of soft-shelled clam Mya arenaria.

In a preferred embodiment, the isolated polynucleotide comprises the cDNA sequence of SEQ ID NO: 1, or a portion thereof, or an antisense polynucleotide.

In a further preferred embodiment, the isolated polynucleotide comprises the RNA sequence of SEQ ID NO: 2, or a portion thereof, or an antisense polynucleotide.

The present invention also provides for an isolated nucleic acid comprising preferably at least 15 consecutive nucleotides which hybridizes to consecutive nucleotides of a retroelement deoxyribonucleic acid or ribonucleic acid present in a mollusk. The mollusk can include, but is not limited to, clams, oysters, scallops, mussels, snails, and soft-shelled clams. In a preferred embodiment, the mollusk is the species of soft-shelled clam Mya arenaria.

In one or more embodiments the consecutive nucleotides of the retroelement deoxyribonucleic acid have a sequence identical to or complementary to a sequence which is about 99, about 98, about 97, about 96, about 95 about 94, about 93, about 92, about 91 or about 90 percent identical to a portion of the sequence set forth in SEQ ID NO: 1.

In one or more embodiments the consecutive nucleotides of the retroelement deoxyribonucleic acid have a sequence identical to or complementary to all or a portion of the sequence set forth in SEQ ID NO: 1.

In one or more embodiments the consecutive nucleotides of the retroelement ribonucleic acid have a sequence identical to a sequence which is about 99, about 98, about 97, about 96, about 95 about 94, about 93, about 92, about 91 or about 90 percent identical to a portion of the sequence set forth in SEQ ID NO: 2.

In one or more embodiments the consecutive nucleotides of the retroelement ribonucleic acid have a sequence identical to or complementary to all or a portion of the sequence set forth in SEQ ID NO: 2.

The further embodiment of the present invention is a polynucleotide that encodes for the steamer retroelement polypeptide. The polypeptide can comprise the sequence of SEQ ID NO: 3, as well as homologues, derivatives, and fragments, especially those due to the degeneracy of the genetic code.

In one or more embodiments consecutive nucleotides of the mollusk retroelement have a sequence identical to all or at least a portion of a sequence which encodes a Gag-Pol precursor polypeptide.

In one or more embodiments consecutive nucleotides of the mollusk retroelement have a sequence identical to all or at least a portion of a sequence which encodes a Gag polypeptide.

In one or more embodiments consecutive nucleotides of the mollusk retroelement have a sequence identical to all or at least a portion of a sequence which encodes a Pol polypeptide.

In one or more embodiments consecutive nucleotides of the mollusk retroelement have a sequence identical to all or at least a portion of a sequence which encodes a polypeptide selected from the group consisting of a capsid polypeptide, a matrix polypeptide, a nucleocapsid polypeptide, a protease polypeptide, an integrase polypeptide, a reverse transcriptase polypeptide or an RNase H polypeptide; or a portion thereof.

The present invention also includes recombinant constructs comprising the DNA comprising the nucleotide sequence of the steamer retroelement or SEQ ID NO: 1, or the antisense DNA comprising the nucleotide sequence of steamer retroelement or SEQ ID NO: 1 or fragments thereof, and a vector, that can be expressed in a transformed host cell. The present invention also includes the host cells transformed with the recombinant construct comprising DNA comprising the nucleotide sequence of the steamer retroelement, or SEQ ID NO: 1, or the antisense DNA comprising the nucleotide sequence of steamer retroelement, or SEQ ID NO: 1 or fragments thereof, and a vector.

Such DNA sequences, no matter how obtained, are useful in the methods set forth herein.

The isolated polynucleotides of the current invention can be used for probes and primers. These probes and primers can be used to detect the steamer element in a mollusk, as well as identify haemic neoplasia in a mollusk. It is also contemplated by the invention that these probes and primers can be used to detect leukemia, leukemia-like disease, and/or other neoplasia in other organisms. The nucleic acids can also be used for basic research tools for the study of haemic neoplasia as well as neoplasia, leukemia and tumors in other organisms.

### Probes and Primers

Further embodiments of the present invention include probes and primers comprising some or all of the DNA comprising the nucleotide sequence of SEQ ID NO: 1, and probes comprising some or all of the DNA with the antisense nucleotide sequence of SEQ ID NO: 1.

Further embodiments of the present invention include probes and primers comprising some or all of the RNA comprising the nucleotide sequence of SEQ ID NO: 2, and probes comprising some or all of the RNA comprising the antisense nucleotide sequence of SEQ ID NO: 2.

In one or more embodiments the nucleic acid has a sequence selected from the group consisting of the sequences set forth in SEQ ID NO: 4-SEQ ID NO: 33.

In particular, primers comprising the nucleotide sequence selected from the group consisting of the sequences set forth in SEQ ID NO: 4-SEQ ID NO: 33, and more preferably selected from the group consisting of the sequences set forth in SEQ ID NO: 20, SEQ ID NO: 21, SEQ ID NO: 24, and SEQ ID NO: 25 are contemplated by the invention.

Other probes and primers contemplated by the present invention can be made by any method known in the art, including the procedures outlined below using in particular the sequence of SEQ ID NO: 1.

In standard nucleic acid hybridization assays, probe must be is labeled in some way, and must be single stranded. Oligonucleotide probes are short (typically 15-50 nucleotides) single-stranded pieces of DNA made by chemical synthesis: mononucleotides are added, one at a time, to a starting mononucleotide, conventionally the 3′ end nucleotide, which is bound to a solid support. Generally, oligonucleotide probes are designed with a specific sequence chosen in response to prior information about the target DNA. Oligonucleotide probes are often labeled by incorporating a ³²P atom or other labeled group at the 5′ end.

Conventional DNA probes are isolated by cell-based DNA cloning or by PCR. In the former case, the starting DNA may range in size from 0.1 kb to hundreds of kilobases in length and is usually (but not always) originally double-stranded. PCR-derived DNA probes have often been less than 10 kb long and are usually, but not always, originally double-stranded.

DNA probes are usually labeled by incorporating labeled dNTPs during an in vitro DNA synthesis reaction by many different methods including nick-translation, random primed labeling, PCR labeling or end-labeling.

Labels can be radioisotopes such as ³²P, ³³P, ³⁵S and ³H, which can be detected specifically in solution or, more commonly, within a solid specimen, such as autoradiography. ³²P has been used widely in Southern blot hybridization, and dot-blot hybridization.

Nonisotopic labeling systems which use nonradioactive probes can also be used in the current invention. Two types of non-radioactive labeling include direct nonisotopic labeling, such as one involving the incorporation of modified nucleotides containing a fluorophore. The other type is indirect nonisotopic labeling, usually featuring the chemical coupling of a modified reporter molecule to a nucleotide precursor. After incorporation into DNA, the reporter groups can be specifically bound by an affinity molecule, a protein or other ligand which has a very high affinity for the reporter group. Conjugated to the latter is a marker molecule or group which can be detected in a suitable assay. This type of labeling would include biotin-streptavidin and digoxigenin.

Primers for use in the various assays of the present invention are also an embodiment of the present invention. Primers useful for the methods of the present invention are also contemplated by the invention and can be prepared by method known in the art as outlined below, using the sequences of the SEQ ID NOs: 1 and 2.

The specificity of amplification depends on the extent to which the primers can recognize and bind to sequences other than the intended target DNA sequences. For complex DNA sources, it is often sufficient to design two primers about 20 nucleotides long. This is because the chance of an accidental perfect match elsewhere in the genome for either one of the primers is extremely low, and for both sequences to occur by chance in close proximity in the specified direction is normally exceedingly low. Although conditions are usually chosen to ensure that only strongly matched primer-target duplexes are stable, spurious amplification products can nevertheless be observed. This can happen if one or both chosen primer sequences contain part of a repetitive DNA sequence, and primers are usually designed to avoid matching to known repetitive DNA sequences, including large runs of a single nucleotide

After the primers are added to denatured template DNA, they bind specifically to complementary DNA sequences at the target site. In the presence of a suitably heat-stable DNA polymerase and DNA precursors (the four deoxynucleoside triphosphates, dATP, dCTP, dGTP and dTTP), they initiate the synthesis of new DNA strands which are complementary to the individual DNA strands of the target DNA segment, and which will overlap each other.

### Method of Using Nucleic Acids—Detection of Steamer Element, Haemic Neoplasia and Other Diseases

The nucleic acids can be used to detect the steamer element in a mollusk. Because the steamer element has been linked to the haemic neoplasia, the detection of the steamer element can also be used to detect and identify HN in a mollusk, including but not limited to, clams, oysters, scallops, mussels, snails, and soft-shelled clams. In a preferred embodiment, the mollusk is the species of soft-shelled clam Mya arenaria.

Additionally, because the steamer element has been shown to be homologous to other cancer-causing retroelements, the nucleic acids can also be used to detect and identify tumors and neoplasia in other organisms.

Because for the nucleic acids of the present invention set forth for the first time a biomarker for disease in mollusks, it can now be used to conduct large-scale screening of populations for mollusks effectively and inexpensively using the methods set forth below.

Any method known in the art can be used to detect the presence or absence of the steamer retroelement. Preferred methods that can be utilized in this analysis are sequencing, hybridization with probes including Southern blot analysis and dot blot analysis, polymerase chain reaction (PCR), PCR with melting curve analysis, PCR with mass spectrometry, fluorescent in situ hybridization, DNA microarrays, single-strand conformation analysis, and restriction length polymorphism analysis. Some of these procedures are exemplified in Examples 4-6.

In some cases, a threshold level is obtained using the same assay and detecting binding to the nucleic acid to a sample from a healthy control, e.g., a mollusk without HN, and if the level of signal is above the threshold level, then the subject would have the steamer retroelement and HN. In one embodiment, the level of the nucleic acid in the subject is about two-fold greater than the threshold level, in a further embodiment, it is about five-fold greater than the threshold level, and in a further embodiment, it is about ten-fold greater than the threshold level.

When a probe is to be used to detect the presence of the steamer element, the biological sample that is to be analyzed must be treated to extract the nucleic acids. The nucleic acids to be targeted usually need to be at least partially single-stranded in order to form a hybrid with the probe sequence. It the nucleic acid is single stranded, no denaturation is required. However, if the nucleic acid to be probed is double stranded, denaturation must be performed by any method known in the art.

The nucleic acid to be analyzed and the probe are incubated under conditions which promote stable hybrid formation of the target sequence in the probe and the target sequence in the nucleic acid. The desired stringency of the hybridization will depend on factors such as the uniqueness of the probe in the part of the genome being targeted, and can be altered by washing procedure, temperature, probe length and other conditions known in the art, as set forth in Maniatis et al. (1982) and Sambrook et al. (1989).

Labeled probes are used to detect the hybrid, or alternatively, the probe is bound to a ligand which labeled either directly or indirectly. Suitable labels and methods for labeling are known in the art, and include biotin, fluorescence, chemiluminescence, enzymes, and radioactivity.

Assays using such probes include Southern blot analysis. In such an assay, a sample is obtained, the DNA processed, denatured, separated on an agarose gel, and transferred to a membrane for hybridization with a probe. Following procedures known in the art (e.g., Sambrook et al. (1989)), the blots are hybridized with a labeled probe and a positive band indicates the presence of the target sequence. The target DNA can also be digested with one or more restriction endonucleases, size-fractionated by agarose gel electrophoresis, denatured and transferred to a nitrocellulose or nylon membrane for hybridization. Following electrophoresis, the test DNA fragments are denatured in strong alkali. As agarose gels are fragile, and the DNA in them can diffuse within the gel, it is usual to transfer the denatured DNA fragments by blotting on to a durable nitrocellulose or nylon membrane, to which single-stranded DNA binds readily. The individual DNA fragments become immobilized on the membrane at positions which are a faithful record of the size separation achieved by agarose gel electrophoresis. Subsequently, the immobilized single-stranded target DNA sequences are allowed to associate with labeled single-stranded probe DNA. The probe will bind only to related DNA sequences in the target DNA, and their position on the membrane can be related back to the original gel in order to estimate their size.

Dot-blot hybridization can also be used. Nucleic acid including genomic DNA, cDNA and RNA is obtained from the subject, denatured and spotted onto a nitrocellulose or nylon membrane and lowed to dry. The membrane is exposed to a solution of labeled single stranded probe sequences and after allowing sufficient time for probe-target heteroduplexes to form, the probe solution is removed and the membrane washed, dried and exposed to an autoradiographic film. A positive spot is an indication of the target sequence in the DNA of the subject and a no spot an indication of the lack of the target sequence in the DNA of the subject.

DNA microarrays can also be used. The surfaces involved are glass rather than porous membranes and similar to reverse dot-blotting, the DNA microarray technologies employ a reverse nucleic acid hybridization approach: the probes consist of unlabeled DNA fixed to a solid support (the arrays of DNA or oligonucleotides) and the target is labeled and in solution.

DNA microarray technology also permits an alternative approach to DNA sequencing by permitting by hybridization of the target DNA to a series of oligonucleotides of known sequence, usually about 7-8 nucleotides long. If the hybridization conditions are specific, it is possible to check which oligonucleotides are positive by hybridization, feed the results into a computer and use a program to look for sequence overlaps in order to establish the required DNA sequence. DNA microarrays have permitted sequencing by hybridization to oligonucleotides on a large scale.

Screening methods of the current invention may involve the amplification of the steamer retroelement. A preferred method for target amplification of nucleic acid sequences is using polymerases, in particular polymerase chain reaction (PCR). PCR or other polymerase-driven amplification methods obtain millions of copies of the relevant nucleic acid sequences which then can be used as substrates for probes or sequenced or used in other assays.

PCR is a rapid and versatile in vitro method for amplifying defined target DNA sequences present within a source of DNA. Usually, the method is designed to permit selective amplification of a specific target DNA sequence(s) within a heterogeneous collection of DNA sequences (e.g. total genomic DNA or a complex cDNA population). To permit such selective amplification, some prior DNA sequence information from the target sequences is required. This information is used to design two oligonucleotide primers (amplimers) which are specific for the target sequence and which are often about 15-25 nucleotides long.

Of particular usefulness in the current invention is the use of oligonucleotide primers to discriminate between target DNA sequences that differ by a single nucleotide in the region of interest called allele-specific PCR. These allele-specific primers will anneal only to the alleles of interest. In this case, the primers of the current invention made from the nucleotide sequence of SEQ ID NO: 1 can be used as a screen of the genomic DNA from the subject. Only if the DNA contains the steamer retroelement will the primers anneal and amplify the product.

Mutation detection using the 5′→3′ exonuclease activity of Taq DNA polymerase (TaqMan™ assay) can also be used as a screening method of the current invention. Such an assay involves hybridization of three primers, the third primer being intended to bind just downstream of one of the conventional primers which should be allele-specific. The additional primer carries a blocking group at the 3′ terminal nucleotide so that it cannot prime new DNA synthesis and at its 5′ end carries a labeled group. In modern versions of the assay, the label is a fluorogenic group and the third primer also carries a quencher group. If the upstream primer which is bound to the same strand is able to prime successfully, Taq DNA polymerase will extend a new DNA strand until it encounters the third primer in which case its 5′→3′ exonuclease will degrade the primer causing release of separate nucleotides containing the dye and the quencher, and an observable increase in fluorescence.

PCR with melting curve analysis can also be used. PCR with melting curve analysis is an extension of PCR where the fluorescence is monitored over time as the temperature changes. Duplexes melt as the temperature increases and the hybridization of both PCR products and probes can be monitored. The temperature-dependent dissociation between two DNA-strands can be measured using a DNA-intercalating fluorophore, such as SYBR green, EvaGreen or fluorophore-labelled DNA probes. In the case of SYBR green (which fluoresces 1000-fold more intensely while intercalated in the minor groove of two strands of DNA), the dissociation of the DNA during heating is measurable by the large reduction in fluorescence that results. Alternatively, juxtapositioned probes (one featuring a fluorophore and the other, a suitable quencher) can be used to determine the complementarity of the probe to the target sequence. This technique is sensitive enough to detect single-nucleotide polymorphisms (SNP) and can distinguish between various alleles by virtue of the dissociation patterns produced.

PCR with mass spectrometry uses mass spectrometry to detect the end product. Primer pairs are used and tagged with molecules of known masses, known as MassCodes. If DNA from any of the agent of primer panel is present, it will be amplified. Each amplified product will carry its specific Masscodes. The PCR product is then purified to remove unbound primers, dNTPs, enzyme and other impurities. Finally, the purified PCR products are subject of ultraviolet as the chemical bond with nucleic acid and primers are photolabile. As the Masscodes are liberated from PCR products they are detected with a mass spectrometer.

Single strand conformation analysis can also be used to determine if the purified and isolated DNA from a subject has particular allele, haplotype or SNP. The conformation of the single-stranded DNA can alter based upon a single base change in the sequence, causing the DNA to migrate differently on electrophoresis. The analysis can involve four steps: (1) polymerase chain reaction (PCR) amplification of DNA sequence of interest; (2) denaturation of double-stranded PCR products; (3) cooling of the denatured DNA (single-stranded) to maximize self-annealing; and (4) detection of mobility difference of the single-stranded DNAs by electrophoresis under non-denaturing conditions. Additionally, the SSCP mobility shifts must be visualized which is done by the incorporation of radioisotope labeling, silver staining, fluorescent dye-labeled PCR primers, and more recently, capillary-based electrophoresis.

### The Steamer Retroelement Protein or Polypeptide

The current invention comprises a novel retroelement denoted as “steamer,” from mollusks, including functional homologues, derivatives, and fragments. The mollusk can include, but is not limited to, clams, oyster, scallops, mussels, snails, and soft-shelled clams. In a preferred embodiment, the mollusk is the species of soft-shelled clam Mya arenaria.

In a preferred embodiment, the retroelement comprises the polypeptide sequence of SEQ ID NO: 3 as well as functional homologues, derivatives, and fragments of the polypeptide comprising SEQ ID NO: 3.

Protein modifications or fragments are contemplated by the current invention. These modifications or fragments are substantially homologous to the primary structural sequence, i.e., amino acid sequence, of the steamer retroelement. Such modifications include but are not limited to acetylation, carboxylation, phosphorylation, glycosylation, ubiquitination, labeling, and various enzymatic modifications known in the art.

Proteins can also be labeled as known in the art and include radioactive isotopes such as ³²P, fluorophores, chemiluminescent agents, enzymes, and antiligands, which serve as binding pair members for labeled ligands.

The present invention also includes biologically active fragments of the polypeptide. Biological activities include ligand-binding, immunological activity, tumorigenic activity, and other biological activity characteristic of the steamer retroelement. Immunological activity includes both immunogenic function in a target immune system and sharing of immunological epitopes for binding, either a competitor or an antigen. An epitope refers to an antigenic determinant of a polypeptide and generally comprises at least three or more amino acids, preferably, five amino acids, and more preferably, 8-10 amino acids.

The present invention also provides for fusion polypeptides and proteins comprising the steamer retroelement and fragments. Fusions may be between two or more polypeptides comprising the steamer retroelement or between the sequences of the steamer retroelement and other polypeptides. The latter fusion proteins would be heterologous and would be constructed to exhibit a combination of properties or activities, such as altered strength or specificity of binding. Fusion partners include, but are not limited to, immunoglobulins, bacterial B-galactosidase, trpE, protein A, B-lactamase, alpha-anylase, alhcole dehydrogenase, and yeast alpha mating factor.

Fusion proteins can be made by either recombinant nucleic acid methods, or be chemically synthesized.

Antibodies

The present invention also provides an antibody directed to a purified mollusk steamer retroelement polypeptide. The mollusk can include, but is not limited to, clams, oysters, scallops, mussels, snails, and soft-shelled clams. In a preferred embodiment, the mollusk is the species of soft-shelled clam Mya arenaria. As would be known in the art, such antibodies would not naturally occur.

In a preferred embodiment, the retroelement comprises the polypeptide sequence of SEQ ID NO: 3 as well as functional homologues, derivatives, and fragments of the polypeptide comprising SEQ ID NO: 3.

The antibodies can be polyclonal or monoclonal antibodies, and fragments thereof, and immunologic binding equivalents thereof, which are capable of binding specifically to the steamer retroelement polypeptide and fragments thereof.

The term “antibody” is used to refer to both a homogenous molecular entity or a mixture such as a serum product made up of a plurality of different molecular entities.

Antibodies, both polyclonal and monoclonal, may be produced by in vitro or in vivo techniques well known in the art. For production of polyclonal antibodies, an appropriate target immune system, typically a rabbit or mouse, is selected, and substantially purified antigen is presented to the immune system in a fashion determined by methods appropriate for the animal and other parameters known by those skilled in the art. The polyclonal antibodies are then purified using techniques known in the art.

Monoclonal antibodies can be made using methods known in the art as well. Appropriate animals again are selected and immunized. After a period of time, the spleens of the animals are excised and the individual spleen cells are fused typically to immortalized myeloma cells under appropriate selection conditions. Then the cells are clonally separated and the supernatant of each clone tested for their production of an appropriate antibody specific for the desired region of antigen.

In one or more embodiments the antibody is directed at a Gag-Pol precursor polypeptide.

In one or more embodiments the antibody is directed at a Gag polypeptide.

In one or more embodiments the antibody is directed at a Pol polypeptide.

In one or more embodiments the antibody is directed at a polypeptide selected from the group consisting of a capsid polypeptide, a matrix polypeptide, a nucleocapsid polypeptide, a protease polypeptide, an integrase polypeptide, a reverse transcriptase polypeptide or an RNase H polypeptide.

In one or more embodiments the antibody is directed at a polypeptide having a sequence identical to a portion of the sequence set forth in SEQ ID NO: 3.

In one or more embodiments the antibody is directed at a polypeptide having a sequence identical to a sequence which is about 99, about 98, about 97, about 96, about 95 about 94, about 93, about 92, about 91 or about 90 percent identical to a portion of the sequence set forth in SEQ ID NO: 3.

### Method of Using Polypeptides-Detection of Steamer Element, Haemic Neoplasia and Other Diseases

The polypeptides can be used to detect the steamer element in a mollusk. Because the steamer element has been linked to the haemic neoplasia, the detection of the steamer element polypeptide or protein can also be used to detect and identify HN in a mollusk. Additionally, because the steamer element has been shown to be homologous to other cancer causing retroelements, the polypeptide can also be used to detect and identify tumors and neoplasia in other organisms.

Because for the steamer element polypeptide of the present invention set forth for the first time a biomarker for disease in mollusks, it can now be used to conduct large-scale screening of populations for mollusks effectively and inexpensively using the methods set forth below. Protein is purified and/or isolated from the biological sample using any method known in the art including but not limited to immunoaffinity chromatography.

Any method known in the art can be used, but preferred methods for detecting increased levels or quantities of the steamer element in a protein sample include quantitative Western blot, immunoblot, quantitative mass spectrometry, enzyme-linked immunosorbent assays (ELISAs), radioimmunoassays (RIA), immunoradiometric assays (IRMA), and immuno enzymatic as says (IEMA) and sandwich assays.

Antibodies are a preferred method of detecting the steamer retroelement polypeptide in a sample. Such antibodies are described above.

In a preferred embodiment, such antibodies will immunoprecipitate the steamer retroelement polypeptide from a solution as well as react with polypeptide on a Western blot, or immunoblot, ELISA, and other assays listed above. In another preferred embodiment, these antibodies will react and detect the steamer retroelement polypeptide in frozen tissue section.

Antibodies for use in these assays can be labeled covalently or non-covalently with an agent that provides a detectable signal. Any label and conjugation method known in the art can be used. Labels, include but are not limited to, enzymes, fluorescent agents, radiolabels, substrates, inhibitors, cofactors, magnetic particles, and chemiluminescent agents.

The levels or quantities of steamer retroelement polypeptide found in a sample are compared to the levels or quantities of the peptide in a healthy control, e.g., haemic neoplasia negative mollusk, and a deviation in the level or quantity of peptides is looked for. This comparison can be done in many ways. The same assay can be performed simultaneously or consecutively, on a purified and/or isolated protein sample from a healthy control and the results compared qualitatively, e.g., visually, i.e., does the protein sample from the healthy control produce the same intensity of signal as the protein sample from the subject in the same assay. In this case, a threshold level is obtained from the same assay with the healthy control and if the level of signal is above the threshold level, then the subject would have the steamer retroelement and HN. In one embodiment, the level of the polypeptide in the subject is about two-fold greater than the threshold level, in a further embodiment, it is about five-fold greater than the threshold level, and in a further embodiment, it is about ten-fold greater than the threshold level.

Alternatively, the results can be compared quantitatively, e.g., a value of the signal for the protein sample from the subject is obtained and compared to a known reference value of the protein in a healthy control. A higher level or quantity of steamer retroelement polypeptide in a sample from a subject as compared to the reference value of the level or quantity of the peptides in a healthy control would indicate the subject has HN or another neoplasm.

### Kits

Screening assays based upon nucleotide testing can also be incorporated into kits. For example, probes and/or primers for the steamer retroelement, reagents for isolating and purifying nucleic acids from the biological sample, reagents for performing assays on the isolated and purified nucleic acid, instructions for use, and comparison sequences could be included in a kit for detection of the steamer retroelement. In particular, a kit could include the primers comprising the sequences set forth in SEQ ID NOs: 4-SEQ ID NO: 33, and most preferably include primers comprising the sequences set forth in SEQ ID NO: 20, SEQ ID NO: 21, SEQ ID NO: 24 and/or SEQ ID NO: 25.

Another kit would test for the steamer retroelement polypeptide and could include antibodies that recognize the peptide of interest, reagents for isolating and/or purifying protein from a sample, reagents for performing assays on the isolated and purified protein, instructions for use, and reference values or the means for obtaining reference values for the quantity or level of peptides in a control sample.

### The Use of the Steamer Retroelement for Research Tools

The steamer retroelement nucleotides, polypeptides, antibodies, gene constructs, and host cells disclosed herein can be used as the basis for drug screening assays and research tools.

In one embodiment, the DNA or RNA comprising the steamer retroelement or SEQ ID NOs: 1 or 2 is contacted with an agent, and a complex between the DNA or RNA and the agent is detected by methods known in the art. One such method is labeling the DNA or RNA and then separating the free DNA or RNA from that bound to the agent. If the agent binds to the DNA or RNA, the agent would be considered a potential therapeutic.

A further embodiment of the present invention is a gene construct comprising the steamer retroelement or SEQ ID NOs: 1 or 2, and a vector. Sequences can be amplified prior to cloning. These gene constructs can be used for testing of therapeutic agents as well as basic research regarding HN and leukemia and other neoplasia.

Such basic research regarding HN would include whether a gene construct comprising the steamer retroelement DNA or RNA could cause disease in a disease-free animal upon transfection or transmission of the DNA or RNA to the animal. Other research regarding HN and other leukemia-like illnesses would include contacting the constructs with environmental triggers and looking for an increase in expression of the steamer element RNA or DNA. Such triggers would include, but are not limited to, extreme temperature and pollutants.

These gene constructs can also be used to transform host cells can be transformed by methods known in the art.

The resulting transformed cells can be used for testing for therapeutic agents as well as basic research regarding HN and leukemia and other neoplasia. Specifically, the host cells can be incubated and/or contacted with a potential therapeutic agent. The resulting expression of the gene construct can be detected and compared to the expression of the gene construct in the cell before contact with the agent.

The expression of the transcripts in host cells can be detected and measured by any method known in the art. The DNA can also be linked to other genes with measurable phenotypes. Expression of the gene linked to the steamer retroelement or SEQ ID NOs: 1 or 2, can be measured before and after the contact with a potential therapeutic agent, as well as a naturally occurring peptide or molecule. Such constructs include but are not limited to a dual luciferase reporter gene or a GFP reporter gene.

These gene constructs as well as the host cells transformed with these gene constructs can also be the basis for transgenic animals for testing both as research tools and for therapeutic agents. Such animals would include but are not limited to, mollusks and nude mice. Phenotypes can be correlated to the genes and looked at in order to determine the genes effect on the animals as well as the change in phenotype after administration or contact with a potential therapeutic agent.

Again basic research regarding the causes of HN and whether the steamer retroelement is a cause or effect of the disease can be performed using the transformed cells and transgenic animals. Such cells and animals can be simply monitored for signs of the disease phenotype, or contacted with an environmental trigger and then monitored for the disease phenotype.

Additionally, the steamer retroelement polypeptide can be used in drug screening assays, free in solution, or affixed to a solid support. All of these forms can be used in binding assays to determine if agents being tested form complexes with the peptides, proteins or fragments, or if the agent being tested interferes with the formation of a complex between the peptide or protein and a known ligand.

Thus, the present invention provides for methods and assays for screening agents, comprising contacting or incubating the test agent with a steamer retroelement polypeptide or a polypeptide comprising SEQ ID NO: 3, and detecting the presence of a complex between the polypeptide and the agent or the presence of a complex between the polypeptide and a ligand, by methods known in the art. In such competitive binding assays, the polypeptide or fragment is typically labeled. Free polypeptide is separated form that in the complex, and the amount of free or uncomplexed polypeptide is measured. This measurement indicates the amount of binding of the test agent to the polypeptide or its interference with the binding of the polypeptide to a ligand.

Antibodies to the steamer retrooelement polypeptide can also be used in competitive drug screening assays. The antibodies compete with the agent being tested for binding to the polypeptide. The antibodies can be used to find agents that have antigenic determinants on the polypeptides, which in turn can be used to develop monoclonal antibodies that target the active sites of the polypeptides.

The invention also provides for polypeptides to be used for rational drug design where structural analogs of biologically active polypeptides can be designed. Such analogs would interfere with the polypeptide in vivo, such as by non-productive binding to target. In this approach the three-dimensional structure of the protein is determined by any method known in the art including but not limited to x-ray crystallography, and computer modeling. Information can also be obtained using the structure of homologous proteins or target-specific antibodies.

Using these techniques, agents can be designed which act as inhibitors or antagonists of the polypeptides, or act as decoys, binding to target molecules non-productively and blocking binding of the active polypeptide.

### EXAMPLES

The present invention may be better understood by reference to the following non-limiting examples, which are presented in order to more fully illustrate the preferred embodiments of the invention. They should in no way be construed to limit the broad scope of the invention.

### Example 1

### Mya Arenaria Collection, Diagnoses of Disease, Samples for Molecular Analysis and Hemocyte Cultures

Mya arenaria were collected and evaluated for leukemia during two surveys in 2009 and two in 2010 (n=100-150 per site per survey). The clams were dug at various high and low-intensity potato farming estuaries around Price Edward Island as previously described in Muttray et al. (2012). For a second survey in 2009 and for the 2010 surveys, sample collection transects were established through the Dunk and Wilmot estuaries (13.6-42% potato farming) from near-field, through mid-field, to far-field sites. M. arenaria were hand dug at low tide and transported to a field laboratory as previously described in Muttray et al. (2012). All samples were processed within 24 hours of collection.

Clams were screened for disease status by withdrawing 0.1 ml of hemolymph from the posterior adductor muscle in a dry sterile 1 milliliter syringe fitted with a sterile 23 gauge needle. The exterior of the clam was wiped with a tissue soaked in 70% ethanol prior to insertion of the needle. A single drop of hemolymph was placed on a microscope slide and left to settle for 5 minutes before examination using a phase-contrast microscope (Leica DMLS 400× magnification). Visual screening was consistently conducted by the same team member, during each survey. Based upon the apparent cell density and shape of hemocytes (small and rounded, absence of appendages), each clam was designated as either “normal” (no leukemic hemocytes, N), “moderate” (20-50% leukemic hemocytes, M), or “heavily leukemic” (>50% leukemic hemocytes, HL) (Muttray et al. (2012)). The diagnosis of HL was confirmed by cytology.

Samples for molecular analysis were obtained by pelleting hemocytes in a refrigerated centrifuge for 5 minutes at 9,600×g. Supernatants were discarded and the remaining pellets were resuspended in RNAlater (Invitrogen) and stored at 4° C. for transportation after which they were stored at −18° C.

Hemocyte cultures were performed on hemocytes from HL and N clams using the method of Walker et al. (2009). The surface of the claim was wiped with ethanol and the remainder of the hemolymph was removed as it was for the diagnosis. The hemolymph was added to 10 milliliters of sterile Walker's medium at room temperature. The hemocytes were then sedimented by centrifugation at 105×g for 10 minutes at 8° C. The “pre-culture supernatant” was transferred to 5 milliliter cryovials and flash frozen in liquid nitrogen. The hemocytes were then gently resuspended in 10 milliliters of Walker's medium and incubated at 8° C. in a tube inverter after which they were sedimented by centrifugation for 8 minutes at 105×g. This was repeated three times for HPL hemocytes after which viability was assessed by Trypan Blue exclusion. The cell suspension was then counted and adjusted to 4−7×10⁴ cells/ml by the addition of Walker's medium. Only contaminant free cell preparations with a viability of greater than 95% were cultured. NHPL hemolymph was added directly to 10 ml of Walker's medium in a 15 ml tissue culture flask and incubated under stationary conditions at 8° C. The HPL cells were transferred to a 125 ml cell reactor/spinner flask and stirred at 32 rpm at 8-10° C. After 12 hours, an aliquot of cell suspension was removed and tested for hemocyte count, viability, and evidence of microbial contamination. The foregoing procedure was repeated after 24 and 48 hours. Upon completion of the incubation period the cell suspension was transferred to sterile 50 ml cell culture tubes and the cells were sedimented by centrifugation at 67×g for 15 minutes at 8° C. The supernatant was transferred to labeled 5 milliliter cryovials (“post-culture supernatant”), flash frozen, and then stored in liquid nitrogen. Sufficient Walker's medium containing 10% (v/v) DMSO was added to the cell pellet to bring the cell count to 4×10⁶ cells/ml. The cell suspension (“cultured cells”) was then transferred to labeled 2 milliliter cryovials, The cyrovials of cell suspension were then placed in a Nalgene “Mr. Frosty Cryo 1° C.” apparatus (ThermoScientific) which was pre-equilibrated to 8° C. The loaded container was placed in dry ice for at least 4 hours after which the frozen cells suspensions were stored in liquid nitrogen.

The loaded container was placed onto dry ice for at least 4 hours after which the frozen cell suspensions were stored in liquid nitrogen. All samples were transported from Prince Edward Island to the CCIW, Burlington, Ontario. Subsequently the frozen cultures were shipped on dry ice to Columbia University, N.Y. Samples of culture medium were flash frozen and stored in liquid nitrogen until returned to CCIW after which they were stored at −80° C. Frozen culture medium and hemocytes in RNAlater were shipped on dry ice and ice respectively from CCIW to Columbia University.

### Example 2

### Hemolymph of Diseased Animals Contains High Levels of Reverse Transcriptase

Cell-free hemolymph (5 μl) from diseased and normal clams as described in Example 1 was assayed for reverse transcriptase activity was determined by incorporation of [³²P]dTTP on a synthetic homopolymer substrate as previously described in Goff et al. (1981). Reactions were performed at 20° C. with poly(rA):oligo(dT) template and Mn++ as divalent cation.

As shown in FIG. 1A, hemolymph from disease clams frequently exhibited high levels of RT activity while healthy controls showed only low background activity. The spot intensity reports the yield of labeled DNA synthesized in vitro.

To confirm that the reverse transcriptase activity was released by neoplastic hemocytes, rather than other tissue, the hemocytes were cultured and the level of reverse transcriptase activity accumulated in the media (5 μl) was determined. As shown in FIG. 1B, the hemocytes from the diseased animals cultured in vitro released high levels of reverse transcriptase into the culture medium, comparable to levels in culture medium from retro-virus infected mammalian cells, while culture medium of hemocytes from healthy animals did not.

Thus, the hemolymph of the diseased animals contains high levels of extracellular reverse transcriptase, suggestive of a retroviral infection.

### Example 2

### Identification of a Novel Retroelement, Steamer

To identify the potential source of the reverse transcriptase activity, the cells from a diseased clam with high RT activity were cultured, total RNA isolated and 454 sequencing of cDNAs used to generate a database of approximately 200,000 sequence reads.

454 sequencing was performed by treating the RNA extracts with DNase I (DNA-free, Ambion, Austin, Tex., USA). cDNA was generated by using the Superscript II system (Invitrogen) for reverse transcription primed by random octamers that were linked to an arbitrary defined 17-mer (5′-GTT TCC CAG TAG GTC TCN NNN NNN N-3′ (SEQ ID NO: 4). The resulting cDNA was treated with RNase H, converted to double stranded DNA template using exoKlenow (NEB) and then randomly amplified by PCR, using a primer corresponding to the defined 17-mer sequence. Products greater than 70 base pairs (bp) were selected by column purification (MinElute, Qiagen, Hilden, Germany) and ligated to specific linkers for sequencing on the 454 Genome Sequencer FLX (454 Life Sciences, Branford, Conn., USA) without template fragmentation (Margulies et al. (2005); Cox-Fisher et al. (2007)). A total of 259,724 reads were obtained. These were clustered using CD-HIT at 98% identity resulting in 77,146 unique reads. The clustered dataset had an average read length of 170 bp and average quality score of 30. The primers and adaptors were trimmed, reads were length-filtered and masked for low complexity regions (WU-BLAST 2.0). A database was generated from the pre-processed reads and searched with Moloney MuLV sequences using BLASTN.

The retroelement-related RNA was cloned using 1 ml of culture medium from Dnear-HL03 cells that was thawed and passed through a 0.45 μm filter, and pelletable material in the filtrate was collected by ultracentrifugation through a 3 ml 20% sucrose cushion for 2 hours at 25,000×g in a SW55 rotor. Total RNA was extracted from the pellet using TRIZOL reagent (Invitrogen). cDNA was generated using 200 ng of RNA and the Super Script First Strand Synthesis system (Invitrogen). Five reads derived of the 454 sequencing with similarity to a retroviral pol gene were selected and the following primers were designed to align with those sequences:

(SEQ ID NO: 5)
C000504-F1 5′gcaagtggtaccacagaggaagtgc3′;
(SEQ ID NO: 6)
57O1-F2 5′cgactgtgcttctggttattggc3′;
(SEQ ID NO: 7)
57O1-F3 5′gcgtttgtaacaccttcaggtgc3′;
(SEQ ID NO: 8)
WX65-F4 5′gcggtgaaaggtgcgttatacctc3′;
(SEQ ID NO: 9)
WX65-R2 5′tgactggcacgcttcacatttcc3′;
(SEQ ID NO: 10)
CX07-F5 5′ccacgtaccctctcgaacttgtatgc3′;
(SEQ ID NO: 11)
C1Q18-R1 5′ggcctaacatgactttgttcgg3′.

PCR reactions were performed using PfuUltra II fusion HS polymerase (Agilent Technologies). The PCR products were TOPO cloned (Invitrogen) and sequenced.

These PCR primers yielded three long overlapping DNA fragments (FIGS. 1C and 1D). FIG. 1C shows the alignment of selected sequences with a retroviral pol gene and FIG. 1D shows the DNAs amplified by the primers identified above.

The sequence of the complete copy of the retroelement containing the fragments was obtained by genome walking using DNA from a healthy animal. To perform genome walking, genomic DNA was extracted, using frozen hemocytes of leukemic and nonleukemic animals were digested with 0.1 mg/ml of proteinase K in digestion buffer (100 mM NaCl, 10 mM Tris-HCl pH 8.0, 25 mM EDTA, 0.5% SDS) at 37° C. overnight, after which phenol-chloroform extraction and DNA precipitation were performed. The DNA was resuspended in buffer TE pH 8.0 and stored at 4° C. Genome walking was performed using Genome Walker Universal kit (Clontech). The primers 5′GW-1 5′ gcagcaagtccaagaagtggggcaaattcg3′ (SEQ ID NO: 12) and 5′GW-1 nested 5′ gtctttgcctgtgtgatctcggtttctg3′ (SEQ ID NO: 13) were designed for a first specific 5′ walk. Once PCR products were cloned and sequenced, the primers 5′GW-2 5′ ggtggaaatgggatcattgaaggaacagc3′ (SEQ ID NO: 14) and 5′GW-2 nested 5′ tggctagtggtattgttgtgggtggggaaa3′ (SEQ ID NO: 15) were designed for a second 5′ walk. For the first 3′ genome walk, the primers 3′GW-1 5′ cgccaccagaagcaaagccatacttca3′ (SEQ ID NO: 16) and 3′GW-1 nested 5′ tcaaccgagcgcagtgtgtgttttg3′ (SEQ ID NO: 17) were designed. Once the PCR products were cloned and sequenced, the primers 3′GW-2 5′ tgctgagccagggacgagtgaccattg3′ (SEQ ID NO: 18) and 3′GW-2 nested 5′ tggtttcccaaacgaggccaaacaaac3′ (SEQ ID NO: 19) were designed for a second 3′ walk. All PCR products were TOPO cloned and sequenced.

The resulting contiguous 4 kb cDNA sequence of a retroelement or retrovirus, was named “steamer” for the common name of the host claim and also by tradition in the transposon field, for a mode of transportation. The sequence is set forth in SEQ ID NO: 1 and has been deposited in GenBank accession number KF319019.

The CCCC/CHCC zinc finger domain is found at nucleotides 956-2055. The DSG PR domain is found at nucleotides 1248-1255. The IADD RT domain is found at 2076-2087. The DAS RNAseH domain is found at nucleotides 2541-2549. The D,D(3,5)E IN domain is found at nucleotides 3402-3563.

| cDNA Sequence of the Steamer Element (SEQ ID NO: 1): | |
|---|---|
| 1 | tgtaacagta ttggctatac taattactat accgtagttt tagtacggtc ccttccgtta |
| 61 | tacttttatg caagagttgg ctcccttgtt tttaaaaaag gacatgcaca ttaaaagtta |
| 121 | tcgtaattga agctacgaag ttgttcaatc attcaacgca taaccgagtt ataaacatgg |
| 181 | tgtcagaagt ggccagagga tcgtaaaggc atgcatctct ctgaaataag cagtcaaatt |
| 241 | gaaacagaag gtaaaagaac attataaacg agcaaagcat cgagccgtga atttccccac |
| 301 | ccacaacaat accactagcc atggctgttc cttcaatgat cccatttcca cctaaacttg |
| 361 | acatggaagg aaacatcagt gacaactgga aaaagttcaa gcgtacgtgg aataactatg |
| 421 | aaatagcggc aggtctcgca gaaaaggatg aaaaactcag aaccgcaact ctattgacat |
| 481 | gcatagggcc agaagccatg gatgtttttg atggatttca ttttgctgaa gagaaagaga |
| 541 | aaactgaaat taaaacagtc attgagaaat ttgagacatt ttgcattgga aaaacaaacg |
| 601 | tcacatatga aaggtacaat tttaatatgt gcacacagac acaggatgaa acatttgaca |
| 661 | cttatgtctc gaggctgaga aaattagtaa agacttgtga gtatgcaaat ctcaccgaga |
| 721 | gcttgattac tgaccgcatt gtcataggta tacgtgagaa cagtgtgcgg aaaagacttc |
| 781 | tgcaagagga taagctaaca cttgacaagt gtattgacat atgcagagct gctgaatcaa |
| 841 | cacaagcaaa ggtcaaatca atgagtggtg caagtggtac cacagaggaa gtgcagtacg |
| 901 | tgaaacaaaa gcaaacgtat agacctaaga caaaaaaccc aacgccaaac ataaataaat |
| 961 | gcaaatattg tggtaaattc tgcacaaaag gtaaatgccc agcctttggg aagaaatgca |
| 1021 | tgaaatgtgg gaaatacaat catttcgcgt ctgaatgtca acaaatagag cagaaaccga |
| 1081 | gatcacacag gcaaagacat gtcagacaat ttgatgttga cgatagttcg gagagtgaga |
| 1141 | atgactttga gattatgaca ttcagcaatg gaacaaggtc caaagttttc gcctccatgc |
| 1201 | ttgtcgtcaa tgttcagaaa acagtaaagt tccaattaga tagtggagca acagcaaacc |
| 1261 | tcattccaaa aacatacgtg ccggaagagc ttattgaatt gaaagcaaat acgcttagaa |
| 1321 | tgtatgacag gtctgagatg aaaacgtatg gtacatgtaa attgacactc aaaaacccaa |
| 1381 | agacttatga cagatacacg gtagagttta tcgttgttga tgacgaattt gccccacttc |
| 1441 | ttggacttgc tgccatccaa agaatgaaac tggtaaaaat ccaatatgaa aacatttgtc |
| 1501 | atgtagaaaa ggaaaatgag ttgcacatgc aagagatcca gaacaattac agtgatgttt |
| 1561 | tccaaggcga aggtactttt gaagaagaac tacatctaga aattgatgat tcggtgactc |
| 1621 | cagtgaaaat gccagtcaga cgtgttccat taggtttaaa agagaaactg aaatgtgaat |
| 1681 | tgcaaagaat ggaaaaagct aacatcatca ccaaagttga aacaccaaca gattgggtat |
| 1741 | ccagcctagt tgtagtaaaa aagccaagtg gtaaattaag aatttgcata gaccccaaac |
| 1801 | cactaaacaa agctcttaaa agaagccact atcccctgcc gatcattgaa gatttactac |
| 1861 | cagaactaag tgaagcaaaa gtcttcagca aatgtgatgt gaaaaatgca ttttggcacg |
| 1921 | tcaaattgga cgaagaatca agttatttaa caacatttga aacgccattc ggacgataca |
| 1981 | gatggaacaa aatgcctttt ggaatctccc cagccccaga atatttccag caatttttag |
| 2041 | agaaaaatct ggaaggacta gatggtgtta aacctatagc ggatgacatt ctaatatatg |
| 2101 | gaaaaggcga aactttccag gacgcagtga aggatcacga cagaaaacta gagaaactgc |
| 2161 | tcaaacggtg taaagagaga aacattaagc tgaacaaaga caaattcgag ttacacaaaa |
| 2221 | cagaaatgcc gttcattgga catctactta cagaaaatgg tgttaagcca gatagtgcaa |
| 2281 | aagttgaagc aatcatgaaa atgcagaaac caagtgacaa gaaagctgtc cagagactgt |
| 2341 | taggagtagt gaattacctc acaaagtttc ttggcaactt gagtgatata tgtgagccta |
| 2401 | tacgcacgct cacacacaag gatgcaatct ggaattggac acatgaacat gacgaagcat |
| 2461 | tcaaaaacat caaaacagca gtgtgcaatg ttccagtcct gagatacttt gactccaggt |
| 2521 | tgaatacagt tctacagtgt gatgcgtcgg aaaccggtct tggtgcgaca ctgatgcaag |
| 2581 | aaggccagcc agtagcatat gcaagcagag cactgacgtc aacggaacag aactacgctc |
| 2641 | aaatagaaaa ggaactactt gctgttgtgt ttggctttga aaaatttcac cagtttacat |
| 2701 | acgggcgccg agtggttgtt gaaagcgacc acaagccatt agaaacgatc agcaagaaag |
| 2761 | cattgcataa agcgccaaag agacttcaaa gaatgctatt aagattacag ctgtacgact |
| 2821 | ttgagatcat ctataagaaa gggaaagaca tgcacattgc tgatactctg tcgagagcgt |
| 2881 | atctacagaa cagttgtgaa agtacaagct taggtgaagt acgttccgtg cagtcagaat |
| 2941 | ttgagaaaga agttgaaacg gtctgtttga cagatttctt agcagtcact ccaagccgtc |
| 3001 | aagagaaaat tagagcagcc acccagctgg atccaacatt agcaatagtt attgagcaaa |
| 3061 | tcaaatgcgg ttggatttcg aaagaaacgc caccagaagc aaagccatac ttcaatattc |
| 3121 | gggatgaact ctctgtagaa aacaacatta tatttcgcgg tgaaaggtgc gttatacctc |
| 3181 | gatgtatgcg cagagacatt ttggaccaaa ttcacacgca cattggggta gaaggatgcc |
| 3241 | tcaaccgagc gcggcagtgt gtgttttggc caaacatgac atctgaaatt aaagatttca |
| 3301 | tagggaaatg tgaagcgtgc cagtcatttg ccagaaagca atgcaaagag ccattgctaa |
| 3361 | accatgatgt accagaccga ccatgggcca aagtcggaac agacattttt accttggatg |
| 3421 | ataataacta cttggtaaca gtcgattact tcagtaattt cttcgagatc gacaaactgg |
| 3481 | aagatatgac atcgcgatgt gtcatcggca aacttaagca acattttgct cgtcatggta |
| 3541 | ttccaaacca gttagtttcg gataatgctc aaacattcaa atcagaaaag ttcaaacagt |
| 3601 | tcactttaca gtgggatttt gaacatgtga cctcatctgc aagataccct caatcgaatg |
| 3661 | gaaaagcaga aagtgcagta aaacgagcaa aatctctcat caaaaagtgt aaacattcac |
| 3721 | atactgaccc aatgttagcc cttttgaacc tgagaaatac ccctctgcag tctacaggat |
| 3781 | acagcccagc tgaacaaagc atgaacaggc agacaagaac actattaccc acaaaagaga |
| 3841 | gtctgctgag gccaaaaacg ctaataaatg tgaaaacaaa tctagacaaa agcaaagcaa |
| 3901 | aacaatcgtt ttactatgac agatcagcaa aacctctgcc aagactagac atgggtacaa |
| 3961 | cagtaagaat caagcctgag aacagtcgag ataaatggga aaaaggcttg attgtcaaca |
| 4021 | gtccgaaaag acgctcatac gatgtaatga cagaaaatgg taccactatc aaccgcaaca |
| 4081 | gaagacatct tcggcaatcg agagagaaat tcactagggc cgacaacgat ccttctgacc |
| 4141 | aaccgagtgg tccggtgcag actgatccta tacccgacct gcagacagat gttgaagcga |
| 4201 | atcggtccaa tactactgct gctgagccag ggacgagtga ccattgtggt ttcccaaacg |
| 4261 | aggccaaaca aactagttct ggacggacag ttaaagttcc gctaagattt aaagattatg |
| 4321 | tgaaataagt cacaagacag tttaggacac ttcactttga gagtgtatca cagtctgata |
| 4381 | agaatccaat cagaaatata tactttaaaa atttagataa gaaagatagt aaggttaagt |
| 4441 | cttgatttaa ttgacaagtg aagcataata catttctata attattttat aagatcctta |
| 4501 | aagagacaaa gtgcttattc aatattccag caccagtgtt aagtgcttag taaagatctt |
| 4561 | tctaggacag ttcttaccac cagactcttt aagtgttaac ttatgtacat attgatagtt |
| 4621 | caaatttatt ttaaatgttc tttaaaggtg attaatctag tcaatagcca taacagactt |
| 4681 | gaactattat gcttatgcgt atcatgtatt tcttgtaaaa tttaaacttc atttcagtgt |
| 4741 | gagattattc cgcagtaagc tttcttacat tcaatgttaa aggaaaaagg atgtaacagt |
| 4801 | attggctata ctaattacta taccgtagtt ttagtacggt cccttccgtt atacttttat |
| 4861 | gcaagagttg gctcccttgt ttttaaaaaa ggacatgcac attaaaagtt atcgtaattg |
| 4921 | aagctacgaa gttgttcaat cattcaacgc ataaccgagt tataaaca |
| RNA Sequence of the Steamer Element derived from the DNA Sequence (SEQ ID NO: 2): | |
| 1 | uguaacagua uuggcuauac uaauuacuau accguaguuu uaguacgguc ccuuccguua |
| 61 | uacuuuuaug caagaguugg cucccuuguu uuuaaaaaag gacaugcaca uuaaaaguua |
| 121 | ucguaauuga agcuacgaag uuguucaauc auucaacgca uaaccgaguu auaaacaugg |
| 181 | ugucagaagu ggccagagga ucguaaaggc augcaucucu cugaaauaag cagucaaauu |
| 241 | gaaacagaag guaaaagaac auuauaaacg agcaaagcau cgagccguga auuuccccac |
| 301 | ccacaacaau accacuagcc auggcuguuc cuucaaugau cccauuucca ccuaaacuug |
| 361 | acauggaagg aaacaucagu gacaacugga aaaaguucaa gcguacgugg aauaacuaug |
| 421 | aaauagcggc aggucucgca gaaaaggaug aaaaacucag aaccgcaacu cuauugacau |
| 481 | gcauagggcc agaagccaug gauguuuuug auggauuuca uuuugcugaa gagaaagaga |
| 541 | aaacugaaau uaaaacaguc auugagaaau uugagacauu uugcauugga aaaacaaacg |
| 601 | ucacauauga aagguacaau uuuaauaugu gcacacagac acaggaugaa acauuugaca |
| 661 | cuuaugucuc gaggcugaga aaauuaguaa agacuuguga guaugcaaau cucaccgaga |
| 721 | gcuugauuac ugaccgcauu gucauaggua uacgugagaa cagugugcgg aaaagacuuc |
| 781 | ugcaagagga uaagcuaaca cuugacaagu guauugacau augcagagcu gcugaaucaa |
| 841 | cacaagcaaa ggucaaauca augaguggug caagugguac cacagaggaa gugcaguacg |
| 901 | ugaaacaaaa gcaaacguau agaccuaaga caaaaaaccc aacgccaaac auaaauaaau |
| 961 | gcaaauauug ugguaaauuc ugcacaaaag guaaaugccc agccuuuggg aagaaaugca |
| 1021 | ugaaaugugg gaaauacaau cauuucgcgu cugaauguca acaaauagag cagaaaccga |
| 1081 | gaucacacag gcaaagacau gucagacaau uugauguuga cgauaguucg gagagugaga |
| 1141 | augacuuuga gauuaugaca uucagcaaug gaacaagguc caaaguuuuc gccuccaugc |
| 1201 | uugucgucaa uguucagaaa acaguaaagu uccaauuaga uaguggagca acagcaaacc |
| 1261 | ucauuccaaa aacauacgug ccggaagagc uuauugaauu gaaagcaaau acgcuuagaa |
| 1321 | uguaugacag gucugagaug aaaacguaug guacauguaa auugacacuc aaaaacccaa |
| 1381 | agacuuauga cagauacacg guagaguuua ucguuguuga ugacgaauuu gccccacuuc |
| 1441 | uuggacuugc ugccauccaa agaaugaaac ugguaaaaau ccaauaugaa aacauuuguc |
| 1501 | auguagaaaa ggaaaaugag uugcacaugc aagagaucca gaacaauuac agugauguuu |
| 1561 | uccaaggcga agguacuuuu gaagaagaac uacaucuaga aauugaugau ucggugacuc |
| 1621 | cagugaaaau gccagucaga cguguuccau uagguuuaaa agagaaacug aaaugugaau |
| 1681 | ugcaaagaau ggaaaaagcu aacaucauca ccaaaguuga aacaccaaca gauuggguau |
| 1741 | ccagccuagu uguaguaaaa aagccaagug guaaauuaag aauuugcaua gaccccaaac |
| 1801 | cacuaaacaa agcucuuaaa agaagccacu auccccugcc gaucauugaa gauuuacuac |
| 1861 | cagaacuaag ugaagcaaaa gucuucagca aaugugaugu gaaaaaugca uuuuggcacg |
| 1921 | ucaaauugga cgaagaauca aguuauuuaa caacauuuga aacgccauuc ggacgauaca |
| 1981 | gauggaacaa aaugccuuuu ggaaucuccc cagccccaga auauuuccag caauuuuuag |
| 2041 | agaaaaaucu ggaaggacua gaugguguua aaccuauagc ggaugacauu cuaauauaug |
| 2101 | gaaaaggcga aacuuuccag gacgcaguga aggaucacga cagaaaacua gagaaacugc |
| 2161 | ucaaacggug uaaagagaga aacauuaagc ugaacaaaga caaauucgag uuacacaaaa |
| 2221 | cagaaaugcc guucauugga caucuacuua cagaaaaugg uguuaagcca gauagugcaa |
| 2281 | aaguugaagc aaucaugaaa augcagaaac caagugacaa gaaagcuguc cagagacugu |
| 2341 | uaggaguagu gaauuaccuc acaaaguuuc uuggcaacuu gagugauaua ugugagccua |
| 2401 | uacgcacgcu cacacacaag gaugcaaucu ggaauuggac acaugaacau gacgaagcau |
| 2461 | ucaaaaacau caaaacagca gugugcaaug uuccaguccu gagauacuuu gacuccaggu |
| 2521 | ugaauacagu ucuacagugu gaugcgucgg aaaccggucu uggugcgaca cugaugcaag |
| 2581 | aaggccagcc aguagcauau gcaagcagag cacugacguc aacggaacag aacuacgcuc |
| 2641 | aaauagaaaa ggaacuacuu gcuguugugu uuggcuuuga aaaauuucac caguuuacau |
| 2701 | acgggcgccg agugguuguu gaaagcgacc acaagccauu agaaacgauc agcaagaaag |
| 2761 | cauugcauaa agcgccaaag agacuucaaa gaaugcuauu aagauuacag cuguacgacu |
| 2821 | uugagaucau cuauaagaaa gggaaagaca ugcacauugc ugauacucug ucgagagcgu |
| 2881 | aucuacagaa caguugugaa aguacaagcu uaggugaagu acguuccgug cagucagaau |
| 2941 | uugagaaaga aguugaaacg gucuguuuga cagauuucuu agcagucacu ccaagccguc |
| 3001 | aagagaaaau uagagcagcc acccagcugg auccaacauu agcaauaguu auugagcaaa |
| 3061 | ucaaaugcgg uuggauuucg aaagaaacgc caccagaagc aaagccauac uucaauauuc |
| 3121 | gggaugaacu cucuguagaa aacaacauua uauuucgcgg ugaaaggugc guuauaccuc |
| 3181 | gauguaugcg cagagacauu uuggaccaaa uucacacgca cauuggggua gaaggaugcc |
| 3241 | ucaaccgagc gcggcagugu guguuuuggc caaacaugac aucugaaauu aaagauuuca |
| 3301 | uagggaaaug ugaagcgugc cagucauuug ccagaaagca augcaaagag ccauugcuaa |
| 3361 | accaugaugu accagaccga ccaugggcca aagucggaac agacauuuuu accuuggaug |
| 3421 | auaauaacua cuugguaaca gucgauuacu ucaguaauuu cuucgagauc gacaaacugg |
| 3481 | aagauaugac aucgcgaugu gucaucggca aacuuaagca acauuuugcu cgucauggua |
| 3541 | uuccaaacca guuaguuucg gauaaugcuc aaacauucaa aucagaaaag uucaaacagu |
| 3601 | ucacuuuaca gugggauuuu gaacauguga ccucaucugc aagauacccu caaucgaaug |
| 3661 | gaaaagcaga aagugcagua aaacgagcaa aaucucucau caaaaagugu aaacauucac |
| 3721 | auacugaccc aauguuagcc cuuuugaacc ugagaaauac cccucugcag ucuacaggau |
| 3781 | acagcccagc ugaacaaagc augaacaggc agacaagaac acuauuaccc acaaaagaga |
| 3841 | gucugcugag gccaaaaacg cuaauaaaug ugaaaacaaa ucuagacaaa agcaaagcaa |
| 3901 | aacaaucguu uuacuaugac agaucagcaa aaccucugcc aagacuagac auggguacaa |
| 3961 | caguaagaau caagccugag aacagucgag auaaauggga aaaaggcuug auugucaaca |
| 4021 | guccgaaaag acgcucauac gauguaauga cagaaaaugg uaccacuauc aaccgcaaca |
| 4081 | gaagacaucu ucggcaaucg agagagaaau ucacuagggc cgacaacgau ccuucugacc |
| 4141 | aaccgagugg uccggugcag acugauccua uacccgaccu gcagacagau guugaagcga |
| 4201 | aucgguccaa uacuacugcu gcugagccag ggacgaguga ccauuguggu uucccaaacg |
| 4261 | aggccaaaca aacuaguucu ggacggacag uuaaaguucc gcuaagauuu aaagauuaug |
| 4321 | ugaaauaagu cacaagacag uuuaggacac uucacuuuga gaguguauca cagucugaua |
| 4381 | agaauccaau cagaaauaua uacuuuaaaa auuuagauaa gaaagauagu aagguuaagu |
| 4441 | cuugauuuaa uugacaagug aagcauaaua cauuucuaua auuauuuuau aagauccuua |
| 4501 | aagagacaaa gugcuuauuc aauauuccag caccaguguu aagugcuuag uaaagaucuu |
| 4561 | ucuaggacag uucuuaccac cagacucuuu aaguguuaac uuauguacau auugauaguu |
| 4621 | caaauuuauu uuaaauguuc uuuaaaggug auuaaucuag ucaauagcca uaacagacuu |
| 4681 | gaacuauuau gcuuaugcgu aucauguauu ucuuguaaaa uuuaaacuuc auuucagugu |
| 4741 | gagauuauuc cgcaguaagc uuucuuacau ucaauguuaa aggaaaaagg auguaacagu |
| 4801 | auuggcuaua cuaauuacua uaccguaguu uuaguacggu cccuuccguu auacuuuuau |
| 4861 | gcaagaguug gcucccuugu uuuuaaaaaa ggacaugcac auuaaaaguu aucguaauug |
| 4921 | aagcuacgaa guuguucaau cauucaacgc auaaccgagu uauaaaca |

### Example 3

### Analysis of the Steamer Element

The amino acid sequences of the conserved regions of the Gag, Protease, RT, RNase H, and IN domains of Steamer were added to an alignment of representative sequences from a database of retrotransposon sequences (Llorens et al (2011)). PhyML 3.0 (Guindon et al. (2010)) was used to generate a maximum likelihood phylogenetic tree using the LG substitution model with 100 replicates for bootstrap analysis.

The Steamer element contains a single long open reading frame (ORF) with sequence similarity to retroviral Gag and Pol proteins, flanked by 177-bp direct repeats similar to the Long Terminal Repeats (LTRs) of integrated proviral DNAs (FIG. 1E). The region of similarity to Gag includes the Major Homology Region (MHR), the most highly-conserved motif of retroviral capsid proteins (Craven et al. (1995)), and a nucleocapsid domain with two zinc fingers containing CCCC and CCHC motifs. The Pol region includes similarities to the retroviral protease with diagnostic DSG active site motif (Loeb et al. (1989)); a reverse transcriptase with a polymerase domain containing an IADD (“YxDD”) box (Yuki et al. (1986)) as well as an RNAse H domain with a diagnostic DG/AS box (Kanaya et al. (1990)); and an integrase with a HHCC zinc finger and a characteristic D,D(3,5), E motif (Kulkosky et al. (1992)). There is no stop codon separating the Gag and Pol ORFs and no ORF similar to an envelope protein. The element contains a primer binding site (PBS) complementary to the 3′ end of the Leu (CAG codon) tRNA of the purple sea urchin (Chan and Lowe (2009)), suggesting that Leu tRNA likely functions as the primer for minus strand DNA synthesis, and a polypurine tract (PPT) sequence serving as primer for plus strand DNA synthesis (Sorge and Hughes (1982)). A maximum likelihood phylogenetic tree (Guindon et al. (2010)), constructed using representative retrotransposon amino acid sequences (Llorens et al. (2011)) and the Gag, protease, RT and integrase domains of Steamer, indicated that Steamer is a member of the Mag lineage of retrotransposons (Michaille et al. (1990)), a subset of the larger family of gypsy/Ty3 elements (Llorens et al. (2011)), with closest similarity to the sea urchin retrotransposon SURL (Springer et al. (1991); Gonzalez and Lessios (1999)) (FIG. 2).

Protein Sequence encoded by steamer Open Reading Frame (SEQ ID NO: 3):
MAVPSMIPFPPKLDMEGNISDNWKKFKRTWNNYEIAAGLAEKDEKLRTATLLTCIGPEA
MDVFDGFHFAEEKEKTEIKTVIEKFETFCIGKTNVTYERYNFNMCTQTQDETFDTYVSRL
RKLVKTCEYANLTESLITDRIVIGIRENSVRKRLLQEDKLTLDKCIDICRAAESTQAKVKS
MSGASGTTEEVQYVKQKQTYRPKTKNPTPNINKCKYCGKFCTKGKCPAFGKKCMKCG
KYNHFASECQQIEQKPRSHRQRHVRQFDVDDSSESENDFEIMTFSNGTRSKVFASMLVV
NVQKTVKFQLDSGATANLIPKTYVPEELIELKANTLRMYDRSEMKTYGTCKLTLKNPKT
YDRYTVEFIVVDDEFAPLLGLAAIQRMKLVKIQYENICHVEKENELHMQEIQNNYSDVF
QGEGTFEEELHLEIDDSVTPVKMPVRRVPLGLKEKLKCELQRMEKANIITKVETPTDWV
SSLVVVKKPSGKLRICIDPKPLNKALKRSHYPLPIIEDLLPELSEAKVFSKCDVKNAFWHV
KLDEESSYLTTFETPFGRYRWNKMPFGISPAPEYFQQFLEKNLEGLDGVKPIADDILIYGK
GETFQDAVKDHDRKLEKLLKRCKERNIKLNKDKFELHKTEMPFIGHLLTENGVKPDSAK
VEAIMKMQKPSDKKAVQRLLGVVNYLTKFLGNLSDICEPIRTLTHKDAIWNWTHEHDE
AFKNIKTAVCNVPVLRYFDSRLNTVLQCDASETGLGATLMQEGQPVAYASRALTSTEQ
NYAQIEKELLAVVFGFEKFHQFTYGRRVVVESDHKPLETISKKALHKAPKRLQRMLLRL
QLYDFEIIYKKGKDMHIADTLSRAYLQNSCESTSLGEVRSVQSEFEKEVETVCLTDFLAV
TPSRQEKIRAATQLDPTLAIVIEQIKCGWISKETPPEAKPYFNIRDELSVENNIIFRGERCVI
PRCMRRDILDQIHTHIGVEGCLNRARQCVFWPNMTSEIKDFIGKCEACQSFARKQCKEPL
LNHDVPDRPWAKVGTDIFTLDDNNYLVTVDYFSNFFEIDKLEDMTSRCVIGKLKQHFAR
HGIPNQLVSDNAQTFKSEKFKQFTLQWDFEHVTSSARYPQSNGKAESAVKRAKSLIKKC
KHSHTDPMLALLNLRNTPLQSTGYSPAEQSMNRQTRTLLPTKESLLRPKTLINVKTNLD
KSKAKQSFYYDRSAKPLPRLDMGTTVRIKPENSRDKWEKGLIVNSPKRRSYDVMTENG
TTINRNRRHLRQSREKFTRADNDPSDQPSGPVQTDPIPDLQTDVEANRSNTTAAEPGTSD
HCGFPNEAKQTSSGRTVKVPLRFKDYVK

### Example 4

### Expression of Steamer RNA is Elevated in Diseased Hemocytes

To test for expression of Steamer RNA transcripts, total RNA was isolated from hemocytes of normal (n=43) and moderately (n=10) and heavily leukemic (n=21) individuals, as described in Example 1, and the levels of Steamer RNA were determined by quantitative RT-PCR (qRTPCR) and normalized to a housekeeping RNA.

To perform qRT-PCR, RNA was extracted from hemocytes conserved in RNAlater using TRIZOL reagent according to the manufacturer's instructions and treated with RNase free DNaseI (Invitrogen). cDNA was generated using 500 ng of RNA and the SuperScriptIII First-Strand Synthesis SuperMix for qRT-PCR kit (Invitrogen) according to instructions. 1 μl of cDNA was used in each of the qPCR reactions to detect Steamer RNA with the FastStart Universal SYBR Green Master (Rox) kit (Roche) using the primers clamRT-F 5′ tgcgtcggaaaccggtcttgg3′ (SEQ ID NO: 20) and clamRT-R 5′ caaccactcggcgcccgtat3′ (SEQ ID NO: 21), or to detect EF1 mRNA using the primers clamEF1F 5′ gaaggatgagggaaaagaggg3′ (SEQ ID NO: 22) and clamEF1R 5′ cacattttcctgctatggtgc3′ (SEQ ID NO: 23) (Siah et al. (2011)). The levels of Steamer mRNA were calculated using a standard curve and expressed as relative to the EF1 mRNA levels. The levels of Steamer RNA in normal and heavily leukemic clams were compared using two-tailed T test and the GraphPad Prism6 program.

Steamer RNA levels were generally low in the normal and moderately leukemic animals, though spanning a large range, and occasional examples were found with high expression (FIG. 3). A large proportion of the highly leukemic samples showed enormously high levels of expression, many fold above the healthy controls. The average level of expression in the diseased animals was about 27-fold above that in the normal, and the mean levels of Steamer RNA strongly correlated with disease status (p<0.0005.) The data were consistent with animals showing sporadic induction of RNA at times during the progression of disease, with periods of very high levels of expression occurring with increasing frequency in more advanced disease.

### Example 5

### Steamer DNA Copy Number is Massively Elevated in Diseased Hemocytes

The high levels of Steamer RNAs in leukemic hemocytes raised the possibility that retroelement-encoded gene products with RT and integrase functions might be available to mediate active reverse transcription and transposition of Steamer DNAs. To test for the presence of reverse transcribed DNAs, total DNA from normal and leukemic clams as described in Example 1 were examined for Steamer sequences by Southern blotting.

To perform Southern blotting analysis, Mya arenaria genomic DNA (20 μg) was digested with the restriction endonucleases BamHI, DraI or HindIII (5 U/μg DNA) for 2 hours at 37° C., followed by addition of 5 more units of enzyme and incubation overnight. Digested DNA was precipitated and resuspended in 25 μl of TE buffer pH 8.0. DNAs (15 μg/lane) were separated by electrophoresis in a 0.7% agarose gel. After ethidium bromide staining DNAs were denatured in alkaline transfer buffer (0.4 M NaOH, 1 M NaCl) and transferred to a nylon membrane. The membrane was neutralized by incubation with neutralization solution (0.5 M Tris-HCl pH 7.2, 1 M NaCl) and prehybridized for 1 h at 42° C. in ULTRAhyb (Ambion).

The probe was obtained by PCR from heavily leukemic genomic DNA using the primers Clamprobe-F 5′ cctgccgatcattgaagatttactacc3′ (SEQ ID NO: 24) and Clamprobe-R 5′ agttgccaagaaactttgtgagg3′ (SEQ ID NO: 25), 30 ng of the probe were labeled using {α-³²P}dCTP and the Prime-It II Random Primer Labeling Kit (Agilent Technologies). Hybridization in ULTRAhyb with the labeled probe was performed at 42° C. for 20 hours. After 2 washes with 2×SSC, 0.1% SDS for 5 min at 42° C. and 2 washes with 0.1×SSC, 0.1% SDS for 15 min at 42° C., the membrane was exposed to X-ray film or to Typhoon plate, exposing for 3 hours.

Restriction digests of DNA from hemocytes of several healthy clams with BamHI to produce 5′ junction fragments of Steamer (FIG. 4A) revealed a small number of bands (2-4) of uniform intensity and varying sizes, suggestive of a low copy number of elements per genome present at highly polymorphic sites (FIG. 4B). DNA from hemocytes of a leukemic animal revealed an intense smear of heterogeneous fragments, indicative of many new, randomly integrated copies. Digests of normal DNA with DraI predicted to release an internal Steamer fragment yielded a single major product of the expected size with only a few other fragments, indicating that most of the copies were intact and homogeneous.

Digestion of leukemic DNA yielded an intense band at the expected size, as well as a number of other fainter fragments, suggesting that most of the newly acquired copies were also intact.

Additional digests of DNAs from two normal and three diseased animals with KpnI, again predicted to release an internal fragment, were examined with similar results (FIG. 4C). The patterns were consistent with the presence of a low copy number of elements endogenous to the genome of healthy animals, and the appearance of a large number of newly integrated Steamer DNAs in diseased cells.

Digests were also performed with additional enzymes to confirm the predicted structure of the DNAs in both normal and diseased animals (FIGS. 5A and B). DNAs were blotted and hybridized with either of two probes from distinct regions of the element (probes 1, 2; FIG. 5A). In all cases, digests predicted to release internal fragments yielded DNA fragments of the expected sizes, suggesting general homogeneity of sequence and close identity to the cloned Steamer DNA. Digests probed so as to detect junction fragments produced small number of bands in normal DNA, and an intense smear indicative of heterogeneous integrations of many copies of the element in diseased DNA (FIG. 5B).

To quantify the Steamer DNA copy number, qPCR reactions were carried out with genomic DNA, using the same primer pairs as in qRT-PCR. 25 ng of genomic DNA was used per reaction in triplicate. Copy number of RT and EF1 was determined by a standard curve using a single plasmid containing both a full length copy of Steamer and the clam EF1 fragment cloned from WfarNM01 DNA. DNA from mantle tissue of healthy clams gave a signal of about 2 copies per haploid genome, consistent with the findings from the Southern blots. DNAs from hemocytes of diseased animals, assayed either as primary cells (n=4) or after culturing (n=3), yielded copy numbers ranging from 100-200 (Table 1).

The combined Southern and qPCR data suggest that Steamer is an extraordinarily active retrotransposon in diseased animals, and undergoes massive expansion and integration into the soft shell clam genome in tumor cells.

TABLE 1
Steamer DNA copy number determined by qPCR performed
with genomic DNA from the indicated individual clams
diagnosed as normal (N) or leukemic (Y).

| | | | Steamer DNA copies |
|---|---|---|---|
| Clam | | | per haploid genome |
| sample ID | Leukemia | DNA Source | (RTseq/EF1) |
| Wfar NM01 | N | Mantle tissue | 2 |
| Dnear 430 | N | Hemocytes | 4 |
| Dnear 07 | Y | Hemocytes | 122 |
| Dnear 08 | Y | Hemocytes | 128 |
| Dnear HL03 | Y | Hemocytes | 96 |
| Dfar 488 | Y | Hemocytes | 143 |
| Dnear HL02 | Y | Cultured Hemocytes | 115 |
| Dnear 426 | Y | Cultured Hemocytes | 172 |
| Dnear 439 | Y | Cultured Hemocytes | 141 |

### Example 6

### Structure of Steamer DNAs

To determine the structure of the Steamer DNAs, inverse PCR was used to amplify the Steamer integration sites in genomic DNA. As shown in FIG. 6A, genomic DNA was digested with MfeI (cleaving only in the flanking DNA), circularized by ligation, and redigested with NsiI at internal sites (N), and finally PCR was performed with outward-directed LTR primers.

Inverse PCR was performed with genomic DNA from mantle tissue (WfarNM01) or leukemic hemocytes (Dnear08 and DnearHL03) extracted (DNeasy Kit, Qiagen Valencia, Calif.) and 125 ng was first digested overnight with 2.5 U of MfeI-HF (NEB, Ipswich, Mass.) at 37° C., which does not cut in the Steamer element. Digested DNA was ligated with T4 DNA ligase in a 25 μl reaction for 20 min at room temperature, heat inactivated for 10 min at 65° C., and digested for 4 hours at 37° C. with 5 U of NsiI (NEB), which cuts four times in the Steamer element. DNA was purified (PCR purification kit, Qiagen) and integration junctions were amplified with PfuUltra II Fusion HS polymerase using primers in the Steamer LTRs (ClamLTR-F2, 5′ acatgcacattaaaagttatcg3′ (SEQ ID NO: 26) and ClamLTR-R1, 5′ ttagtatagccaatactgttac3′(SEQ ID NO: 27)). The PCR protocol consisted of incubations at 95° C. for 2 minutes, followed by 35 cycles of 95° C. for 20 seconds, 50° C. for 20 seconds, and 68° C. for 5 minutes, with a final extension at 72° C. for 5 minutes. Inverse PCR products were analyzed on an agarose gel, isolated by gel extraction of specific bands or PCR purification of the whole PCR product (Qiagen), and cloned using the Zero Blunt TOPO cloning kit (Life Technologies). DNA sequences of the inserts in individual cloned plasmids were determined using flanking M13F and M13R primers. The integration sites were confirmed by a diagnostic PCR using ClamLTR-F2 and a reverse primer in the genomic DNA flanking the corresponding integration site (enSR6 5′ tccagccatgtgttcctgct3′ (SEQ ID NO: 28); IMDL8c1R 5′ aactccaatacccttcaatt3′ (SEQ ID NO: 29); IMDL8c6R 5′ agctgtctagattggaagtg3′ (SEQ ID NO: 30); IMHL03c2R 5′ attgtcccagattcacagat3′ (SEQ ID NO: 31); and IMHL03c3R 5′ gtaggtcttatacatttgag3′ (SEQ ID NOS: 32)). For these reactions 100 ng of DNA was used with Taq polymerase at 95° C. for 5 minutes, followed by 35 cycles of 95° C. for 30 seconds, 50° C. for 30 seconds, and 72° C. for 30 seconds, with a final extension of 72° C. for 5 minutes (products are approximately 150 bp each).

The complete endogenous Steamer sequence was amplified from normal clam genomic DNA (WfarNM01) with primers enSR6 and enSF1 5′ cgcagggatcaatagacgacac3′ (SEQ ID NO: 33) as shown SEQ ID NO: 1.

DNA of a healthy clam yielded a single major PCR product of an authentic integration site (FIG. 6B). The DNA sequence of this product revealed integration site junctions corresponding to the predicted LTR 5′ and 3′ ends, and a 5 bp direct repeat flanking the integration site (FIG. 6C).

Inverse PCR of two diseased animals amplified a large number of integration sites, and 5-10 were cloned and sequenced from each animal (examples shown in FIG. 6C). Further PCR reactions using primers in the Steamer LTR and the flanking genomic sequence revealed that the single integration site found in the normal animal was present in all three animals. Diagnostic primers designed for two integration sites from each diseased animal revealed that both diseased animals contained all four of the novel integration sites, while the normal animal contained none. Thus, Steamer has inserted at multiple new sites in genomic DNA of leukemic clams, most likely by somatic retrotransposition, and may exhibit a preference for common integration sites that were utilized in independent leukemias.

### Example 7

### Identification and Analysis of Steamer Transcripts and Proteins

Using simple Northern blots of RNAs from diseased tissues the transcripts produced from the element are identified. Sequencing of cDNAs derived with carefully chosen primers is used to obtain complete structures.

The protein products encoded by the element are determined by expressing portions of the ORFs in E. coli, and generating polyclonal antisera in rabbits against the partially purified proteins. Antiserum against the steamer RT, Gag, all the Pol domains, and Env products identified are obtained.

Monoclonal antibodies from mouse hybridomas are prepared to provide cleaner reagents and eliminate concern for long-term availability. The sera is used in Western blots of diseased tissue lysates; for histochemistry of diseased tissues; and for rapid diagnosis of specimens both in the field and in the laboratory.

The serum is used to explore the expression and processing of the polyproteins; Gag and Pol products are cleaved into a small number of mature proteins, corresponding to the MA, CA, NC, PR, RT, and IN proteins. The presence of less common products for which there are precedents such as a dUTPase, or a transforming oncogene such as the cyclins of the piscine viruses, is investigated.

### Example 8

### Characterization of Steamer Polypeptides

Characterization of the reverse transcriptase activity is performed using the recombinant protein from E. coli, validated with limited material from tissues. DNA polymerase and RNase H activities also are characterized and their optimum pH, salt, temperature, and divalent ion requirements are determined to facilitate future screens of samples for the presence of the virus. These studies further define the processivity and error rate of the polymerase.

Detection of the virus in explanted hemocyte cultures from diseased specimens and propagation of the virus in cultures of normal hemocytes from healthy animals are attempted. The presence of free virus is a controversial one, generally dismissed by the field, with efforts to confirm positive sightings (Oprandy et al. (1983))) having almost universally failed (AboElkhair et al. (2012)). However, due to the present invention, there are now reagents that will allow the detection of the virions with much greater sensitivity, and firmly confirm or dismiss these reports. Whether virus can infect cells in culture to induce the expression of viral gene products is determined.

Explanted hemocytes for these experiments are maintained in Walker medium, relatively conventional medium, used to culture both hemolymph and cultured hemocytes from diseased animals.

Infected cells and infectious DNA copies of the genome in culture supernatants of mammalian cells transfected with the viral DNA is used to investigate infection of healthy cell cultures with exogenous cell-free virus, or by cell-cell contact via coculture with infected cells.

Virion particles are characterized by their biochemical properties. Their repertoire of viral proteins are detected with our antisera; their RNA content are determined by RT-PCR and Northern blots; and their isopycnic density on sucrose gradients is measured. Their structure and morphology are analyzed by transmission electron microscopy. Sections of infected cells are examined for budding virions or for intracellular virion particles (by analogy to IAPs, intracellular A-type particles (Mietz et al. (1987)).

Genetic transfer and retroviral transduction of mollusk cells in culture have been achieved (Boulo et al. (1986); Boulo et al. (2000); Jordan et al. (1988)).

### Example 9

### Regulation of Viral Gene Expression

Cell types or tissues of the diseased animals express the highest levels of viral mRNAs and protein are determined by measuring RNA by Q-PCR and viral proteins by Western blot of preparations of various tissues. In situ hybridization and immunostaining of histological sections of whole-mounts also are used to provide a better overview of the tissue distribution.

Whether viral RNAs and proteins are expressed at higher levels after explanting hemocytes from diseased animals into culture, and whether any such expression continues over the lifetime of the cell cultures is determined.

### Example 10

### Induced Activity of Steamer Retrovirus

Whether virus expression is increased by various treatments, such as reagents that induce DNA damage e.g. etoposides, ionizing radiation or UV exposure; reagents that affect DNA methylation e.g. 5-AzaCytosine, BrUdR or IUdR, potent inducers of endogenous retrovirus expression in mammalian cells (and perhaps even in clams: (Oprandy and Chang (1983)); and the environmental toxins that are considered possible initiators of the HN disease in the wild, such as PCB mixtures and pesticides is determined.

Whether the viral promoter responds to temperature shifts, including heat shock, or to other stressors such as oxidative stress e.g. hydrogen peroxide, is determined. These experiments are enormously facilitated by engineering a GFP or luciferase reporter construct in which the viral promoter is placed upstream of the reporter ORF. These studies help define the conditions and circumstances under which the virus is activated or induced.

### Example 11

### Whether “Steamer” is a Cause or Contributor to the HN Disease is Investigated

There is evidence provided herein of a strong correlation of the virus with disease (FIGS. 5A and B). It is asked whether the virus is a consequence or can directly induce disease.

Whether infection of hemocytes in culture causes changes in morphology, DNA content (ploidy), or changes in growth properties of the cells are determined using the traditional reporters of transformation in mammalian cells induced by the frankly oncogenic viruses: changes in visible cell morphology, minimal conditions for growth (serum requirement), maximum cell density, rate of growth, cell cycle status as determined by PI stain/flow cytometry, rate of apoptosis, and survival lifetime in culture.

Whether infection leads to polyploid, to date the most consistent correlate of HN (Cooper et al. (1982); DeVera et al. (2005)), is determined. Changes in p53, p63, and p73 levels and intracellular localization (Jessen-Eller et al. (2002)), and changes in mortalin, a gene product that modulates p53 localization (Walker et al. (2011)) are characterized.

Relocalization of these tumor suppressor proteins upon infection is consistently seen in the authentic tumor cells.

Induction of expression of the cell surface protein detected by the 1e10 monoclonal reagent is a marker of the leukemic cells in authentic HN (Miosky et al. (1989); Reinisch et al. (1984); Smolowitz et al. (1993); Walker et al. (1993)). Infection with steamer can elicit these aspects of HN, suggesting that steamer might indeed be a contributor to disease and not merely a correlate of disease.

### REFERENCES

AboElkhair et al. 2012. Lack of detection of a putative retrovirus associated with haemic neoplasia in the soft shell clam Mya arenaria. J. Invertebr. Pathol. 109:97-104.
AboElkhair et al. 2009. Reverse transcriptase activity associated with haemic neoplasia in the soft-shell clam Mya arenaria. Dis. Aquat. Organ. 84:57-63.
AboElkhair et al. 2009a. Reverse transcriptase activity in tissues of the soft shell clam Mya arenaria affected with haemic neoplasia. J. Invertebr. Pathol. 102:133-140.
Barber 2004. Neoplastic diseases of commercially important marine bivalves. Aquat. Living Resour. 17:449-466.
Barker et al. 1997. Detection of mutant p53 in clam leukemia cells. Exp. Cell Res. 232:240-245.
Beere and Green. 2001. Stress management—heat shock protein-70 and the regulation of apoptosis. Trends Cell Biol. 11:6-10.
Bottger et al. 2008. Genotoxic stress-induced expression of p53 and apoptosis in leukemic clam hemocytes with ctyoplasmically sequestered p53. Cancer Res. 68:777-782.
Boulo et al. 1996. Transient expression of luciferase reporter gene after lipofection in oyster (Crassostrea gigas) primary cell cultures. Mol. Mar. Biol. Biotechnol. 5:167-174.
Boulo et al. 2000. Infection of cultured embryo cells of the pacific oyster, Crassostrea gigas, by pantropic retroviral vectors. In Vitro Cell. Dev. Biol. Anim. 36:395-399.
Brasset et al. 2006. Viral particles of the endogenous retrovirus ZAM from Drosophila melanogaster use a pre-existing endosome/exosome pathway for transfer to the oocyte. Retrovirology 3:25.
Brown et al. 1977. Prevalence of neoplasia in 10 New England populations of the soft-shell claim (Mya arenaria). Ann. NY Acad. Sci. 298:522-534.
Chalvet et al. 1999. Proviral amplification of the Gypsy endogenous retrovirus of Drosophila melanogaster involves env-independent invasion of the female germline. The EMBO journal 18(9):2659-2669.
Chan and Lowe 2009. GtRNAdb: a database of transfer RNA genes detected in genomic sequence. Nucleic acids research 37 (Database issue):D93-97.
Collins and Mulcahy 2003. Cell-free transmission of a haemic neoplasm in the cockle Cerastoderma edule. Dis. Aquat. Organ. 54(1):61-67.
Cooper et al. 1982. The course and mortality of a hematopoietic neoplasm in the soft-shell clam, Mya arenaria. J. Invertebr. Pathol. 39:149-157.
Cooper and Chang. 1982. Accuracy of blood cytological screening techniques for the diagnosis of a possible hematopoetic neoplasm in the bivalve mollusk, Mya arenaria. J. Invertebr. Pathol. 39:281-289.
Cox-Foster et al. 2007. A metagenomic survey of microbes in honey bee colony collapse disorder. Science 318(5848):283-287.
Craven et al. 1995 Genetic analysis of the major homology region of the Rous sarcoma virus

Gag protein. Journal of Virology 69(7):4213-4227.
De Vera et al. 2005. Occurrence of Hemic Neoplasia in Slipper Oyster, Crassostrea iredalei (Faustino, 1928), in Dagupan City, Philippines, p. 321-325. In P. Walker, R. Lester, and M. G. Bondad-Reantaso (ed.), Diseases in Asian Aquaculture V.
Delaporte et al. 2008. Immunophenotyping of Mya arenaria neoplastic hemocytes using propidium iodide and a specific monoclonal antibody by flow cytometry. J. Invertebr. Pathol. 99:120-122.
Eaton and Kent. 1992. A retrovirus in chinook salmon (Oncorhynchus tshawytscha) with plasmacytoid leukemia and evidence for the etiology of the disease. Cancer Research 52:6496-6500.
Elston et al. 1988. Progression, lethality and remission of hemic neoplasia in the bay mussel Mytilis edulis. Dis. Aquat. Organ. 4:135-142.
Elston et al. 1988. Transmission of hemic neoplasia in the bay mussel, Mytilus edulis, using whole cells and cell homogenate. Dev. Comp. Immunol. 12:719-727.
Elston et al. 1992. Disseminated neoplasia of bivalve mollusks. Rev. Aquat. Sci. 6:405-466.
Farley 1969. Probable neoplastic disease of the hematopoietic system in oysters Crassostrea virginica and Crassostra gigas. Natl. Cancer Insti. Monogr. 31:541-555.
Farley et al. 1986. New occurrence of epizootic sarcoma in Chesapeake Bay soft-shell clams, Mya arenaria. Fishery Bull. 84:851-857.
Goff et al. 1981. Isolation and properties of Moloney murine leukemia virus mutants: use of a rapid assay for release of virion reverse transcriptase. Journal of Virology 38(1):239-248
Gonzalez and Lessios (1999) Evolution of sea urchin retroviral-like (SURL) elements: evidence from 40 echinoid species. Molecular Biology and Evolution 16(7):938-952.
Guindon et al. 2010. New algorithms and methods to estimate maximum-likelihood phylogenies: assessing the performance of PhyML 3.0. Syst. Biol. 59(3):307-321.
Hart et al. 1996. Complete nucleotide sequence and transcriptional analysis of snakehead fish retrovirus. Journal of Virology 70:3606-3616.
Holbrook et al. 2009. Soft-shell clam (Mya arenaria) p53: A structural and functional comparison to human p53. Gene 433:81-87.
House et al. 1998. Soft shell clams Mya arenaria with disseminated neoplasia demonstrate reverse transcriptase activity. Dis. Aquat. Organ. 34:187-192.
Inaki and Liu. 2012. Structural mutations in cancer: mechanistic and functional insights. Trends in Genetics 28(11):550-559.
Jessen-Eller et al. 2002. A new invertebrate member of the p53 gene family is developmentally expressed and responds to polychlorinated biphenyls. Environ. Health Perspect. 110:377-385.
Jordan et al. 1998. Pantropic retroviral vectors mediate somatic cell transformation and expression of foreign genes in dipteran insects. Insect Mol. Biol. 7:215-222.
Kanaya et al. 1990. Identification of the amino acid residues involved in an active site of Escherichia coli ribonuclease H by site-directed mutagenesis. The Journal of Biological Chemistry 265(8):4615-4621.
Kelley et al. 2001. Expression of homologues for p53 and p73 in the softshell clam (Mya arenaria), a naturally occurring model for human cancer. Oncogene 20:748-758.
Kim et al. 1994. Retroviruses in invertebrates: the gypsy retrotransposon is apparently an infectious retrovirus of Drosophila melanogaster. PNAS 91(4):1285-1289.
Krishnakumar et al. 1999. Environmental contaminants and the prevalence of hemic neoplasia (leukemia) in the common mussel (Mytilus edulis complex) from Puget Sound, Washington, U.S.A. J. Invertebr. Pathol. 73:135-146.
Kulkosky et al. (1992) Residues critical for retroviral integrative recombination in a region that is highly conserved among retroviral/retrotransposon integrases and bacterial insertion sequence transposases. Molecular and Cellular Biology 12(5):2331-2338.
Landsberg. 1996. Neoplasia and biotoxins in bivalves: is there a connection? J. Shellfish Res. 15:203-230.
LaPierre et al. 1998. Walleye retroviruses associated with skin tumors and hyperplasias encode cyclin D homologs. Journal of Virology 72:8765-8771.
Levin (2002) Newly identified retrotransposons of the Ty3/gypsy class in Fungi, Plants, and vertebrates. Mobile DNA II, eds Craig N L, Craigie R, Gellert M, & Lambowitz AM (ASM Press, Washington, D.C.), pp 684-701.
Llorens et al. (2011) The Gypsy Database (GyDB) of mobile genetic elements: release 2.0. Nucleic Acids Research 39 (Database issue):D70-74.
Loeb et al. 1989. Mutational analysis of human immunodeficiency virus type 1 protease suggests functional homology with aspartic proteinases. Journal of Virology 63(1):111-121.
Lowe and Moore. 1978. Cytology and quantitative cytochemistry of a proliferative atypical hemocytic condition in Mytilus edulis (Bivalvia, mollusca). J. Natl. Cancer Inst. 60:1455-1459.
Maniatis et al. (1982) Sambrook et al. (1989) (1989) Molecular Cloning: A Laboratory Manual (Cold Spring Harbor Laboratory, 2nd Ed., Cold Spring Harbor, N. Y.
Margulies et al. 2005 Genome sequencing in microfabricated high-density picoliter reactors. Nature 437(7057):376-380.
McLaughlin et al. (1992) Transmission studies of sarcoma in the soft-shell clam, Mya arenaria. In Vivo 6(4):367-370.
Medina et al. 1993. Isolation of infectious particles having reverse transcriptase activity and producing hematopoietic neoplasia in Mya arenaria. J. Shellfish Res. 12:112-113.
Michaille et al. (1990) The complete sequence of mag, a new retrotransposon in Bombyx mori. Nucleic Acids Research 18(3):674.
Mietz et al. 1987. Nucleotide sequence of a complete mouse intracisternal A-particle genome: relationship to known aspects of particle assembly and function. Journal of Virology 61:3020-3029.
Miosky et al. 1989. Leukemia cell specific protein of the bivalve mollusk Mya arenaria. J. Invertebr. Pathol. 53:32-40.
Morrison et al. 1993. Disseminated sarcomas of soft-shell clams, Mya arenaria Linnaeus 1758, from sites in Nova Scotia and New Brunswick. J. Shellfish Res. 12:65-69.
Muttray et al. 2012 Haemocytic leukemia in Prince Edward Island (PEI) soft shell clam (Mya arenaria): Spatial distribution in agriculturally impacted estuaries. Sci. Total Environ. 424:130-142.
Muttray et al. 2008. Invertebrate p53-like mRNA isoforms are differentially expressed in mussel haemic neoplasia. Mar. Environ. Res. 66:412-421.
Oprandy et al. 1981. Isolation of a viral agent causing hematopoietic neoplasia in the soft-shell clam Mya arenaria. J. Invertebr. Pathol. 34:45-51.
Oprandy and Chang. 1983. 5-bromodeoxyuridine induction of hematopoietic neoplasia and retrovirus activation in the soft-shell clam, Mya arenaria. J. Invertebr. Pathol. 42:196-206.
Pariseau et al. 2009. Potential link between exposure to fungicides chlorothalonil and mancozeb and haemic neoplasia development in the soft-shell clam Mya arenaria: a laboratory experiment. Mar. Pollut. Bull. 58(4):503-514.
Reinisch et al. 1984. Epizootic neoplasia in softshell clams collected from New Bedford Harbor. J. Hazardous Wastes 1:73-77.
Reinisch et al. 1983. Unique antigens on neoplastic cells of the soft shell clam Mya arenaria. Dev. Comp. Immunol. 7:33-39.
Romalde et al. 2007. Evidence of retroviral etiology for disseminated neoplasia in cockles (Cerastoderma edule). J. Invertebr. Pathol. 94(2):95-101.
Reno et al. 1994. Flow cytometry and chromosome analysis of Softshell clams, Mya arenaria, with disseminated neoplasia. J. Invertebr. Pathol. 64:163-172.
Rovnak and uackenbush. 2010. Walleye dermal sarcoma virus: molecular biology and oncogenesis. Viruses 2:1984-1999.
Sambrook et al. (1989) Molecular Cloning: A Laboratory Manual (Cold Spring Harbor Laboratory, 2nd Ed., Cold Spring Harbor, N.Y.
Siah et al. 2011. Induction of transposase and polyprotein RNA levels in disseminated neoplastic hemocytes of soft-shell clams: Mya arenaria. Dev. Comp. Immunol. 35:151-154.
Siah et al. (2013) Transcriptome analysis of neoplastic hemoctyes in soft-shell clams Mya arenaria: Focus on cell-cycle molecular mechanism. Results in Immunology 3:95-103.
Schneider (2008) Heat stress in the intertidal: comparing survival and growth of an invasive and native mussel under a variety of thermal conditions. Biol. Bull. 215(3):253-264.
Smith et al. 2011. Resolving the evolutionary relationships of mollusks with phylogenic tools. Nature 480:364-367.
Smolowitz et al. 1989. Ontogeny of leukemic cells of the soft shell clam. J. Invertebr. Pathol. 53:41-51.
Smolowitz and Reinisch. 1993. A novel adhesion protein expressed by ciliated epithelium, hemocytes, and leukemia cells in soft-shell clams. Dev. Comp. Immunol. 17:475-481.
Solyom et al. 2012. Extensive somatic L1 retrotransposition in colorectal tumors. Genome Research 22(12):2328-2338.
Song et al. 1994. An env-like protein encoded by a Drosophila retroelement: evidence that gypsy is an infectious retrovirus. Genes and Development 8(17):2046-2057.
Sorge and Hughes. 1982. Polypurine tract adjacent to the U3 region of the Rous sarcoma virus genome provides a cis-acting function. Journal of Virology 43(2):482-488.
Springer et al. 1991. Retroviral-like element in a marine invertebrate. PNAS 88(19):8401-8404.
St-Jean et al. 2005. Detecting p53 family proteins in haemocytic leukemia cells of Mytilus edulis from Pictou Harbour, Nova Scotia, Canada. Can J. Fish. Aquat. Sci. 62:2055-2066.
Sunila. 1992. Serum-cell interactions in transmission of sarcoma in the soft shell clam, Mya arenaria L. Comp. Biochem. Physiol. Comp. Physiol. 102:727-730.
Sunila and Farley. 1989. Environmental limits for survival of sarcoma cells from the soft-shell clam Mya arenaria. Dis. Aqua. Org. 7:111-115.
Taraska and Bottger. 2013. Selective initiation and transmission of disseminated neoplasia in the soft shell clam Mya arenaria dependent on natural disease prevalence and animal size. J Invertebr Pathol. 112(1):94-101.
Walker et al. 2006. Mortalin-based cytoplasmic sequestration of p53 in a nonmammalian cancer model. Am J Pathol 168:1526-1530.
Walker et al. 2009. Mass culture and characterization of tumor cells from a naturally occurring invertebrate cancer model: applications for human and animal disease and environmental health. Biol. Bull. 216(1):23-39.
Walker et al. 2011. p53 Superfamily Proteins in Marine Bivalve Cancer and Stress Biology, pp. 1-36, Advances in Marine Biology, vol. 59. Elsevier LTD.
White et al. 1993. The expression of an adhesion-related protein by clam hemocytes. J. Invertebr. Pathol 61:253-259.
Yoshikura et al. 1977. Enhancement of 5-iododeoxyuridine-induced endogenous Ctype virus activation by polycyclic hydrocarbons: apparent lack of parallelism between enhancement and carcinogenicity. J. Natl. Cancer Inst. 58(4):1035-1040.
Yuki et al. 1986. Identification of genes for reverse transcriptase-like enzymes in two Drosophila retrotransposons, 412 and gypsy; a rapid detection method of reverse transcriptase genes using YXDD box probes. Nucleic Acids Research 14(7):3017-3030.

1. An isolated cDNA coding for a retroelement found in mollusks, said cDNA comprising the nucleotide sequence of SEQ ID NO: 1 or functional homologues, derivatives or fragments thereof.

2. The isolated cDNA of claim 1, wherein the mollusk is selected from the group consisting of clams, oysters, scallops, mussels, snails, and soft-shelled clams.

3. The isolated cDNA of claim 1, wherein the mollusk is of the species mya arenaria.

4. The isolated cDNA of claim 1, wherein the cDNA is a fragment of the nucleotide sequence of SEQ ID NO: 1, and comprises at least fifteen nucleotides.

5. An isolated cDNA comprising at least fifteen consecutive nucleotides that specifically hybridizes to the cDNA comprising SEQ ID NO: 1 or functional homologues, derivatives or fragments thereof.

6. The cDNA of claim 5, wherein the nucleotides are selected from the group consisting of the DNA comprising SEQ ID NOs: 4-33.

7. The cDNA of claim 5, wherein the nucleotides are selected from the group consisting of the DNA comprising SEQ ID NO:20, SEQ ID NO: 21, SEQ ID NO: 24, and SEQ ID NO:25.

8. A construct comprising a vector and an isolated cDNA comprising the nucleotide sequence of SEQ ID NO: 1 or functional homologues, derivatives or fragments thereof.

9. A host cell comprising the construct of claim 8.

10. An antibody directed to a retroelement found in mollusks and associated with haemic neoplasia.

11. The antibody of claim 10, wherein the antibody is chosen from the group consisting of monoclonal and polyclonal antibodies.

12. The antibody of claim 10, wherein the mollusk is selected from the group consisting of clams, oysters, scallops, mussels, snails, and soft-shelled clams.

13. The antibody of claim 10, wherein the mollusk is of the species mya arenaria.

14. The antibody of claim 10, wherein the retroelement comprises the polypeptide comprising the amino acid sequence of SEQ ID NO: 3 or functional homologues, derivatives or fragments thereof.

15. A method of identifying or screening for a neoplasia or leukemia in a subject, comprising:
a. obtaining a sample of cells or protein from the subject;
b. contacting the sample with the antibody of directed to a retroelement found in mollusks and associated with haemic neoplasia;
c. detecting any specific binding in step (b); and
d. determining the subject has a neoplasia or leukemia based upon the binding of the antibody with the retroelement in the sample.

16. The method of claim 15, wherein the subject is a mollusk.

17. The method of claim 16, wherein the mollusk is selected from the group consisting of clams, oysters, scallops, mussels, snails, and soft-shelled clams.

18. The method of claim 15, wherein the retroelement comprises the polypeptide comprising the amino acid sequence of SEQ ID NO: 3 or functional homologues, derivatives or fragments thereof.

19. The method of claim 15, wherein the neoplasia is haemic neoplasia.

20. The method of claim 15, further comprising providing a healthy control sample; and contacting the antibody directed to a retroelement found in mollusks and associated with haemic neoplasia to obtain a threshold level, wherein the step of determining that the patient has a neoplasia or leukemia comprises a step of comparing the binding to the threshold level, and wherein the binding is greater than the threshold level, the subject is determined to have a neoplasia or leukemia.

21. A method of identifying or screening for a neoplasia or leukemia in a subject comprising:
a. obtaining a sample of deoxyribonucleic acid or ribonucleic acid from the subject;
b. contacting the sample of step (a) with a nucleic acid that specifically hybridizes with the cDNA of SEQ ID NO: 1, under conditions permitting the nucleic acid to specifically hybridize to a deoxyribonucleic acid or ribonucleic acid encoding a retroelement; and
c. detecting any hybridization in step (b), and
d. determining that the subject has a neoplasia or leukemia based upon the binding of the cDNA with the deoxyribonucleic acid or ribonucleic acid encoding a portion of a retroelement in the sample.

22. The method of claim 21, wherein the subject is a mollusk.

23. The method of claim 22, wherein the mollusk is selected from the group consisting of clams, oysters, scallops, mussels, snails, and soft-shelled clams.

24. The method of claim 21, wherein the neoplasia is haemic neoplasia.

25. The method of claim 21, further comprising providing a healthy control sample; and contacting the cDNA of SEQ ID NO: 1 to obtain a threshold level, wherein the step of determining that the subject has a neoplasia or leukemia comprises a step of comparing the binding to the threshold level, and wherein the binding is greater than the threshold level, the subject is determined to have a neoplasia or leukemia.

26. A method of identifying or screening for a neoplasia or leukemia in a subject, comprising:
a. obtaining biological tissue from the subject;
b. isolating and purifying a sample of nucleic acid from the biological tissue or bodily fluid; and
a. detecting the presence of steamer retroelement in the sample of nucleic acid;
wherein the presence of the steamer retroelement in the sample of nucleic acid is detected by an assay selected from the group consisting of (a) hybridizing a steamer retroelement probe to the nucleic acid sample, and detecting the presence of hybridization products, (b) hybridizing an allele-specific probe to nucleic acid sample and detecting the presence of hybridization products in the sample, (c) amplifying all or part of the steamer retroelement from the nucleic acid sample to produce an amplified sequence and sequencing the amplified sequence, (d) amplifying all or part of the steamer retroelement from the nucleic acid sample using primers for the steamer retroelement and determining the presence of a hybridization product in the sample, (e) amplifying all or part of the steamer retroelement from the nucleic acid sample using primers for the steamer retroelement and determining the presence of amplicons in the sample, (f) molecularly cloning all or part of the steamer retroelement from the nucleic acid sample to produce a cloned sequence and sequencing the cloned sequence, (f) amplification of steamer retroelement sequences in the nucleic acid sample and hybridization of the amplified sequences to nucleic acid probes which comprise the steamer retroelement and (g) in situ hybridization of the nucleic acid sample with nucleic acid probes which comprise the steamer retroelement;
wherein the presence of steamer retroelement determines, or identifies the subject as having neoplasia or leukemia.

27. The method of claim 26, wherein the subject is a mollusk.

28. The method of claim 27, wherein the mollusk is selected from the group consisting of clams, oysters, scallops, mussels, snails, and soft-shelled clams.

29. The method of claim 26, wherein the neoplasia is haemic neoplasia.

30. A kit to identify or screen for a neoplasia or leukemia in a subject, comprising the isolated cDNA of claim 5, reagents for isolating and purifying nucleic acids from a biological sample, reagents for performing assays on the isolated and purified nucleic acids, and instructions for use.

31. A kit to identify or screen for a neoplasia or leukemia in a subject, comprising the antibody of claim 10, reagents for isolating and purifying protein from a biological sample, reagents for performing assays on the isolated and purified nucleic proteins, and instructions for use.


SEQUENCE LISTING (38 sequences)

SEQ ID NO: 1 - DNA, 4968 bp, Mya arenaria
tgtaacagtattggctatactaattactataccgtagttttagtacggtcccttccgttatacttttatgcaagagttggctcccttgtttttaaaaaaggacatgcacattaaaagttatcgtaattgaagctacgaagttgttcaatcattcaacgcataaccgagttataaacatggtgtcagaagtggccagaggatcgtaaaggcatgcatctctctgaaataagcagtcaaattgaaacagaaggtaaaagaacattataaacgagcaaagcatcgagccgtgaatttccccacccacaacaataccactagccatggctgttccttcaatgatcccatttccacctaaacttgacatggaaggaaacatcagtgacaactggaaaaagttcaagcgtacgtggaataactatgaaatagcggcaggtctcgcagaaaaggatgaaaaactcagaaccgcaactctattgacatgcatagggccagaagccatggatgtttttgatggatttcattttgctgaagagaaagagaaaactgaaattaaaacagtcattgagaaatttgagacattttgcattggaaaaacaaacgtcacatatgaaaggtacaattttaatatgtgcacacagacacaggatgaaacatttgacacttatgtctcgaggctgagaaaattagtaaagacttgtgagtatgcaaatctcaccgagagcttgattactgaccgcattgtcataggtatacgtgagaacagtgtgcggaaaagacttctgcaagaggataagctaacacttgacaagtgtattgacatatgcagagctgctgaatcaacacaagcaaaggtcaaatcaatgagtggtgcaagtggtaccacagaggaagtgcagtacgtgaaacaaaagcaaacgtatagacctaagacaaaaaacccaacgccaaacataaataaatgcaaatattgtggtaaattctgcacaaaaggtaaatgcccagcctttgggaagaaatgcatgaaatgtgggaaatacaatcatttcgcgtctgaatgtcaacaaatagagcagaaaccgagatcacacaggcaaagacatgtcagacaatttgatgttgacgatagttcggagagtgagaatgactttgagattatgacattcagcaatggaacaaggtccaaagttttcgcctccatgcttgtcgtcaatgttcagaaaacagtaaagttccaattagatagtggagcaacagcaaacctcattccaaaaacatacgtgccggaagagcttattgaattgaaagcaaatacgcttagaatgtatgacaggtctgagatgaaaacgtatggtacatgtaaattgacactcaaaaacccaaagacttatgacagatacacggtagagtttatcgttgttgatgacgaatttgccccacttcttggacttgctgccatccaaagaatgaaactggtaaaaatccaatatgaaaacatttgtcatgtagaaaaggaaaatgagttgcacatgcaagagatccagaacaattacagtgatgttttccaaggcgaaggtacttttgaagaagaactacatctagaaattgatgattcggtgactccagtgaaaatgccagtcagacgtgttccattaggtttaaaagagaaactgaaatgtgaattgcaaagaatggaaaaagctaacatcatcaccaaagttgaaacaccaacagattgggtatccagcctagttgtagtaaaaaagccaagtggtaaattaagaatttgcatagaccccaaaccactaaacaaagctcttaaaagaagccactatcccctgccgatcattgaagatttactaccagaactaagtgaagcaaaagtcttcagcaaatgtgatgtgaaaaatgcattttggcacgtcaaattggacgaagaatcaagttatttaacaacatttgaaacgccattcggacgatacagatggaacaaaatgccttttggaatctccccagccccagaatatttccagcaatttttagagaaaaatctggaaggactagatggtgttaaacctatagcggatgacattctaatatatggaaaaggcgaaactttccaggacgcagtgaaggatcacgacagaaaactagagaaactgctcaaacggtgtaaagagagaaacattaagctgaacaaagacaaattcgagttacacaaaacagaaatgccgttcattggacatctacttacagaaaatggtgttaagccagatagtgcaaaagttgaagcaatcatgaaaatgcagaaaccaagtgacaagaaagctgtccagagactgttaggagtagtgaattacctcacaaagtttcttggcaacttgagtgatatatgtgagcctatacgcacgctcacacacaaggatgcaatctggaattggacacatgaacatgacgaagcattcaaaaacatcaaaacagcagtgtgcaatgttccagtcctgagatactttgactccaggttgaatacagttctacagtgtgatgcgtcggaaaccggtcttggtgcgacactgatgcaagaaggccagccagtagcatatgcaagcagagcactgacgtcaacggaacagaactacgctcaaatagaaaaggaactacttgctgttgtgtttggctttgaaaaatttcaccagtttacatacgggcgccgagtggttgttgaaagcgaccacaagccattagaaacgatcagcaagaaagcattgcataaagcgccaaagagacttcaaagaatgctattaagattacagctgtacgactttgagatcatctataagaaagggaaagacatgcacattgctgatactctgtcgagagcgtatctacagaacagttgtgaaagtacaagcttaggtgaagtacgttccgtgcagtcagaatttgagaaagaagttgaaacggtctgtttgacagatttcttagcagtcactccaagccgtcaagagaaaattagagcagccacccagctggatccaacattagcaatagttattgagcaaatcaaatgcggttggatttcgaaagaaacgccaccagaagcaaagccatacttcaatattcgggatgaactctctgtagaaaacaacattatatttcgcggtgaaaggtgcgttatacctcgatgtatgcgcagagacattttggaccaaattcacacgcacattggggtagaaggatgcctcaaccgagcgcggcagtgtgtgttttggccaaacatgacatctgaaattaaagatttcatagggaaatgtgaagcgtgccagtcatttgccagaaagcaatgcaaagagccattgctaaaccatgatgtaccagaccgaccatgggccaaagtcggaacagacatttttaccttggatgataataactacttggtaacagtcgattacttcagtaatttcttcgagatcgacaaactggaagatatgacatcgcgatgtgtcatcggcaaacttaagcaacattttgctcgtcatggtattccaaaccagttagtttcggataatgctcaaacattcaaatcagaaaagttcaaacagttcactttacagtgggattttgaacatgtgacctcatctgcaagataccctcaatcgaatggaaaagcagaaagtgcagtaaaacgagcaaaatctctcatcaaaaagtgtaaacattcacatactgacccaatgttagcccttttgaacctgagaaatacccctctgcagtctacaggatacagcccagctgaacaaagcatgaacaggcagacaagaacactattacccacaaaagagagtctgctgaggccaaaaacgctaataaatgtgaaaacaaatctagacaaaagcaaagcaaaacaatcgttttactatgacagatcagcaaaacctctgccaagactagacatgggtacaacagtaagaatcaagcctgagaacagtcgagataaatgggaaaaaggcttgattgtcaacagtccgaaaagacgctcatacgatgtaatgacagaaaatggtaccactatcaaccgcaacagaagacatcttcggcaatcgagagagaaattcactagggccgacaacgatccttctgaccaaccgagtggtccggtgcagactgatcctatacccgacctgcagacagatgttgaagcgaatcggtccaatactactgctgctgagccagggacgagtgaccattgtggtttcccaaacgaggccaaacaaactagttctggacggacagttaaagttccgctaagatttaaagattatgtgaaataagtcacaagacagtttaggacacttcactttgagagtgtatcacagtctgataagaatccaatcagaaatatatactttaaaaatttagataagaaagatagtaaggttaagtcttgatttaattgacaagtgaagcataatacatttctataattattttataagatccttaaagagacaaagtgcttattcaatattccagcaccagtgttaagtgcttagtaaagatctttctaggacagttcttaccaccagactctttaagtgttaacttatgtacatattgatagttcaaatttattttaaatgttctttaaaggtgattaatctagtcaatagccataacagacttgaactattatgcttatgcgtatcatgtatttcttgtaaaatttaaacttcatttcagtgtgagattattccgcagtaagctttcttacattcaatgttaaaggaaaaaggatgtaacagtattggctatactaattactataccgtagttttagtacggtcccttccgttatacttttatgcaagagttggctcccttgtttttaaaaaaggacatgcacattaaaagttatcgtaattgaagctacgaagttgttcaatcattcaacgcataaccgagttataaaca

SEQ ID NO: 2 - RNA, 4968 nt, Mya arenaria
uguaacaguauuggcuauacuaauuacuauaccguaguuuuaguacggucccuuccguuauacuuuuaugcaagaguuggcucccuuguuuuuaaaaaaggacaugcacauuaaaaguuaucguaauugaagcuacgaaguuguucaaucauucaacgcauaaccgaguuauaaacauggugucagaaguggccagaggaucguaaaggcaugcaucucucugaaauaagcagucaaauugaaacagaagguaaaagaacauuauaaacgagcaaagcaucgagccgugaauuuccccacccacaacaauaccacuagccauggcuguuccuucaaugaucccauuuccaccuaaacuugacauggaaggaaacaucagugacaacuggaaaaaguucaagcguacguggaauaacuaugaaauagcggcaggucucgcagaaaaggaugaaaaacucagaaccgcaacucuauugacaugcauagggccagaagccauggauguuuuugauggauuucauuuugcugaagagaaagagaaaacugaaauuaaaacagucauugagaaauuugagacauuuugcauuggaaaaacaaacgucacauaugaaagguacaauuuuaauaugugcacacagacacaggaugaaacauuugacacuuaugucucgaggcugagaaaauuaguaaagacuugugaguaugcaaaucucaccgagagcuugauuacugaccgcauugucauagguauacgugagaacagugugcggaaaagacuucugcaagaggauaagcuaacacuugacaaguguauugacauaugcagagcugcugaaucaacacaagcaaaggucaaaucaaugaguggugcaagugguaccacagaggaagugcaguacgugaaacaaaagcaaacguauagaccuaagacaaaaaacccaacgccaaacauaaauaaaugcaaauauugugguaaauucugcacaaaagguaaaugcccagccuuugggaagaaaugcaugaaaugugggaaauacaaucauuucgcgucugaaugucaacaaauagagcagaaaccgagaucacacaggcaaagacaugucagacaauuugauguugacgauaguucggagagugagaaugacuuugagauuaugacauucagcaauggaacaagguccaaaguuuucgccuccaugcuugucgucaauguucagaaaacaguaaaguuccaauuagauaguggagcaacagcaaaccucauuccaaaaacauacgugccggaagagcuuauugaauugaaagcaaauacgcuuagaauguaugacaggucugagaugaaaacguaugguacauguaaauugacacucaaaaacccaaagacuuaugacagauacacgguagaguuuaucguuguugaugacgaauuugccccacuucuuggacuugcugccauccaaagaaugaaacugguaaaaauccaauaugaaaacauuugucauguagaaaaggaaaaugaguugcacaugcaagagauccagaacaauuacagugauguuuuccaaggcgaagguacuuuugaagaagaacuacaucuagaaauugaugauucggugacuccagugaaaaugccagucagacguguuccauuagguuuaaaagagaaacugaaaugugaauugcaaagaauggaaaaagcuaacaucaucaccaaaguugaaacaccaacagauuggguauccagccuaguuguaguaaaaaagccaagugguaaauuaagaauuugcauagaccccaaaccacuaaacaaagcucuuaaaagaagccacuauccccugccgaucauugaagauuuacuaccagaacuaagugaagcaaaagucuucagcaaaugugaugugaaaaaugcauuuuggcacgucaaauuggacgaagaaucaaguuauuuaacaacauuugaaacgccauucggacgauacagauggaacaaaaugccuuuuggaaucuccccagccccagaauauuuccagcaauuuuuagagaaaaaucuggaaggacuagaugguguuaaaccuauagcggaugacauucuaauauauggaaaaggcgaaacuuuccaggacgcagugaaggaucacgacagaaaacuagagaaacugcucaaacgguguaaagagagaaacauuaagcugaacaaagacaaauucgaguuacacaaaacagaaaugccguucauuggacaucuacuuacagaaaaugguguuaagccagauagugcaaaaguugaagcaaucaugaaaaugcagaaaccaagugacaagaaagcuguccagagacuguuaggaguagugaauuaccucacaaaguuucuuggcaacuugagugauauaugugagccuauacgcacgcucacacacaaggaugcaaucuggaauuggacacaugaacaugacgaagcauucaaaaacaucaaaacagcagugugcaauguuccaguccugagauacuuugacuccagguugaauacaguucuacagugugaugcgucggaaaccggucuuggugcgacacugaugcaagaaggccagccaguagcauaugcaagcagagcacugacgucaacggaacagaacuacgcucaaauagaaaaggaacuacuugcuguuguguuuggcuuugaaaaauuucaccaguuuacauacgggcgccgagugguuguugaaagcgaccacaagccauuagaaacgaucagcaagaaagcauugcauaaagcgccaaagagacuucaaagaaugcuauuaagauuacagcuguacgacuuugagaucaucuauaagaaagggaaagacaugcacauugcugauacucugucgagagcguaucuacagaacaguugugaaaguacaagcuuaggugaaguacguuccgugcagucagaauuugagaaagaaguugaaacggucuguuugacagauuucuuagcagucacuccaagccgucaagagaaaauuagagcagccacccagcuggauccaacauuagcaauaguuauugagcaaaucaaaugcgguuggauuucgaaagaaacgccaccagaagcaaagccauacuucaauauucgggaugaacucucuguagaaaacaacauuauauuucgcggugaaaggugcguuauaccucgauguaugcgcagagacauuuuggaccaaauucacacgcacauugggguagaaggaugccucaaccgagcgcggcaguguguguuuuggccaaacaugacaucugaaauuaaagauuucauagggaaaugugaagcgugccagucauuugccagaaagcaaugcaaagagccauugcuaaaccaugauguaccagaccgaccaugggccaaagucggaacagacauuuuuaccuuggaugauaauaacuacuugguaacagucgauuacuucaguaauuucuucgagaucgacaaacuggaagauaugacaucgcgaugugucaucggcaaacuuaagcaacauuuugcucgucaugguauuccaaaccaguuaguuucggauaaugcucaaacauucaaaucagaaaaguucaaacaguucacuuuacagugggauuuugaacaugugaccucaucugcaagauacccucaaucgaauggaaaagcagaaagugcaguaaaacgagcaaaaucucucaucaaaaaguguaaacauucacauacugacccaauguuagcccuuuugaaccugagaaauaccccucugcagucuacaggauacagcccagcugaacaaagcaugaacaggcagacaagaacacuauuacccacaaaagagagucugcugaggccaaaaacgcuaauaaaugugaaaacaaaucuagacaaaagcaaagcaaaacaaucguuuuacuaugacagaucagcaaaaccucugccaagacuagacauggguacaacaguaagaaucaagccugagaacagucgagauaaaugggaaaaaggcuugauugucaacaguccgaaaagacgcucauacgauguaaugacagaaaaugguaccacuaucaaccgcaacagaagacaucuucggcaaucgagagagaaauucacuagggccgacaacgauccuucugaccaaccgagugguccggugcagacugauccuauacccgaccugcagacagauguugaagcgaaucgguccaauacuacugcugcugagccagggacgagugaccauugugguuucccaaacgaggccaaacaaacuaguucuggacggacaguuaaaguuccgcuaagauuuaaagauuaugugaaauaagucacaagacaguuuaggacacuucacuuugagaguguaucacagucugauaagaauccaaucagaaauauauacuuuaaaaauuuagauaagaaagauaguaagguuaagucuugauuuaauugacaagugaagcauaauacauuucuauaauuauuuuauaagauccuuaaagagacaaagugcuuauucaauauuccagcaccaguguuaagugcuuaguaaagaucuuucuaggacaguucuuaccaccagacucuuuaaguguuaacuuauguacauauugauaguucaaauuuauuuuaaauguucuuuaaaggugauuaaucuagucaauagccauaacagacuugaacuauuaugcuuaugcguaucauguauuucuuguaaaauuuaaacuucauuucagugugagauuauuccgcaguaagcuuucuuacauucaauguuaaaggaaaaaggauguaacaguauuggcuauacuaauuacuauaccguaguuuuaguacggucccuuccguuauacuuuuaugcaagaguuggcucccuuguuuuuaaaaaaggacaugcacauuaaaaguuaucguaauugaagcuacgaaguuguucaaucauucaacgcauaaccgaguuauaaaca

SEQ ID NO: 3 - protein, 1335 aa, Mya arenaria
MAVPSMIPFPPKLDMEGNISDNWKKFKRTWNNYEIAAGLAEKDEKLRTATLLTCIGPEAMDVFDGFHFAEEKEKTEIKTVIEKFETFCIGKTNVTYERYNFNMCTQTQDETFDTYVSRLRKLVKTCEYANLTESLITDRIVIGIRENSVRKRLLQEDKLTLDKCIDICRAAESTQAKVKSMSGASGTTEEVQYVKQKQTYRPKTKNPTPNINKCKYCGKFCTKGKCPAFGKKCMKCGKYNHFASECQQIEQKPRSHRQRHVRQFDVDDSSESENDFEIMTFSNGTRSKVFASMLVVNVQKTVKFQLDSGATANLIPKTYVPEELIELKANTLRMYDRSEMKTYGTCKLTLKNPKTYDRYTVEFIVVDDEFAPLLGLAAIQRMKLVKIQYENICHVEKENELHMQEIQNNYSDVFQGEGTFEEELHLEIDDSVTPVKMPVRRVPLGLKEKLKCELQRMEKANIITKVETPTDWVSSLVVVKKPSGKLRICIDPKPLNKALKRSHYPLPIIEDLLPELSEAKVFSKCDVKNAFWHVKLDEESSYLTTFETPFGRYRWNKMPFGISPAPEYFQQFLEKNLEGLDGVKPIADDILIYGKGETFQDAVKDHDRKLEKLLKRCKERNIKLNKDKFELHKTEMPFIGHLLTENGVKPDSAKVEAIMKMQKPSDKKAVQRLLGVVNYLTKFLGNLSDICEPIRTLTHKDAIWNWTHEHDEAFKNIKTAVCNVPVLRYFDSRLNTVLQCDASETGLGATLMQEGQPVAYASRALTSTEQNYAQIEKELLAVVFGFEKFHQFTYGRRVVVESDHKPLETISKKALHKAPKRLQRMLLRLQLYDFEIIYKKGKDMHIADTLSRAYLQNSCESTSLGEVRSVQSEFEKEVETVCLTDFLAVTPSRQEKIRAATQLDPTLAIVIEQIKCGWISKETPPEAKPYFNIRDELSVENNIIFRGERCVIPRCMRRDILDQIHTHIGVEGCLNRARQCVFWPNMTSEIKDFIGKCEACQSFARKQCKEPLLNHDVPDRPWAKVGTDIFTLDDNNYLVTVDYFSNFFEIDKLEDMTSRCVIGKLKQHFARHGIPNQLVSDNAQTFKSEKFKQFTLQWDFEHVTSSARYPQSNGKAESAVKRAKSLIKKCKHSHTDPMLALLNLRNTPLQSTGYSPAEQSMNRQTRTLLPTKESLLRPKTLINVKTNLDKSKAKQSFYYDRSAKPLPRLDMGTTVRIKPENSRDKWEKGLIVNSPKRRSYDVMTENGTTINRNRRHLRQSREKFTRADNDPSDQPSGPVQTDPIPDLQTDVEANRSNTTAAEPGTSDHCGFPNEAKQTSSGRTVKVPLRFKDYVK

SEQ ID NO: 4 - DNA, 25 bp, Mya arenaria
  misc_feature (18)..(25): n is a, c, g, or t
gtttcccagtaggtctcnnnnnnnn

SEQ ID NO: 5 - DNA, 25 bp, Mya arenaria
gcaagtggtaccacagaggaagtgc

SEQ ID NO: 6 - DNA, 23 bp, Mya arenaria
cgactgtgcttctggttattggc

SEQ ID NO: 7 - DNA, 23 bp, Mya arenaria
gcgtttgtaacaccttcaggtgc

SEQ ID NO: 8 - DNA, 24 bp, Mya arenaria
gcggtgaaaggtgcgttatacctc

SEQ ID NO: 9 - DNA, 23 bp, Mya arenaria
tgactggcacgcttcacatttcc

SEQ ID NO: 10 - DNA, 26 bp, retroviral provirus
ccacgtaccctctcgaacttgtatgc

SEQ ID NO: 11 - DNA, 22 bp, Mya arenaria
ggcctaacatgactttgttcgg

SEQ ID NO: 12 - DNA, 30 bp, Mya arenaria
gcagcaagtccaagaagtggggcaaattcg

SEQ ID NO: 13 - DNA, 28 bp, Mya arenaria
gtctttgcctgtgtgatctcggtttctg

SEQ ID NO: 14 - DNA, 29 bp, Mya arenaria
ggtggaaatgggatcattgaaggaacagc

SEQ ID NO: 15 - DNA, 30 bp, Mya arenaria
tggctagtggtattgttgtgggtggggaaa

SEQ ID NO: 16 - DNA, 27 bp, Mya arenaria
cgccaccagaagcaaagccatacttca

SEQ ID NO: 17 - DNA, 25 bp, Mya arenaria
tcaaccgagcgcagtgtgtgttttg

SEQ ID NO: 18 - DNA, 27 bp, Mya arenaria
tgctgagccagggacgagtgaccattg

SEQ ID NO: 19 - DNA, 27 bp, Mya arenaria
tggtttcccaaacgaggccaaacaaac

SEQ ID NO: 20 - DNA, 21 bp, Mya arenaria
tgcgtcggaaaccggtcttgg

SEQ ID NO: 21 - DNA, 20 bp, Mya arenaria
caaccactcggcgcccgtat

SEQ ID NO: 22 - DNA, 21 bp, Mya arenaria
gaaggatgagggaaaagaggg

SEQ ID NO: 23 - DNA, 21 bp, Mya arenaria
cacattttcctgctatggtgc

SEQ ID NO: 24 - DNA, 27 bp, Mya arenaria
cctgccgatcattgaagatttactacc

SEQ ID NO: 25 - DNA, 23 bp, Mya arenaria
agttgccaagaaactttgtgagg

SEQ ID NO: 26 - DNA, 22 bp, Mya arenaria
acatgcacattaaaagttatcg

SEQ ID NO: 27 - DNA, 22 bp, Mya arenaria
ttagtatagccaatactgttac

SEQ ID NO: 28 - DNA, 20 bp, Mya arenaria
tccagccatgtgttcctgct

SEQ ID NO: 29 - DNA, 20 bp, Mya arenaria
aactccaatacccttcaatt

SEQ ID NO: 30 - DNA, 20 bp, Mya arenaria
agctgtctagattggaagtg

SEQ ID NO: 31 - DNA, 20 bp, Mya arenaria
attgtcccagattcacagat

SEQ ID NO: 32 - DNA, 20 bp, Mya arenaria
gtaggtcttatacatttgag

SEQ ID NO: 33 - DNA, 22 bp, Mya arenaria
cgcagggatcaatagacgacac

SEQ ID NO: 34 - DNA, 36 bp, Mya arenaria
acgacacacattatttgtacattattgatatgttac

SEQ ID NO: 35 - DNA, 36 bp, Mya arenaria
ttagtgtgtgatggttgtacaatggtcctgaacaac

SEQ ID NO: 36 - DNA, 36 bp, Mya arenaria
catggttctcatgtttgtacaatgttcttcaaagaa

SEQ ID NO: 37 - DNA, 36 bp, Mya arenaria
ttcatgctccaattgtgtacaaattgtttatcaggt

SEQ ID NO: 38 - DNA, 36 bp, Mya arenaria
agcgttcattaaatgtgtacaaaatgaatgcctcat